(19) United States
(12) Patent Application Publication
(10) Pub. No.: US 2022/0332737 A1
(43) Pub. Date: Oct. 20, 2022

(54) CARBOCYCLIC NUCLEOSIDE ANALOGUE

(71) Applicant: Ludwig-Maximilians-Universität München, München (DE)

(72) Inventor: Thomas CARELL, Krailling (DE)

(73) Assignee: Ludwig-Maximilians-Universität München, München (DE)

(21) Appl. No.: 17/616,701

(22) PCT Filed: Jun. 5, 2019
(86) PCT No.: PCT/EP2020/065621

§ 371 Date: Dec. 6, 2021

(30) Foreign Application Priority Data
Jun. 7, 2019 (EP) 19178928.8

(51) Int. Cl. C07F 9/6521; C07D 251/16; C12Q 1/6886

(52) U.S. Cl. CPC C07F 9/6521; C07D 251/16; C12Q 1/6886; C12Q 1/683

(57) ABSTRACT

The present invention relates to novel hydrolytically stable carbon-cyclic 5-aza-2-deoxycytidine and carbocyclic 5-aza-cytidine compounds and pro-drugs thereof as hypomethylating agents.

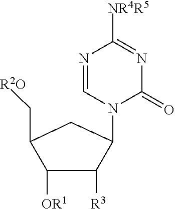

The present invention relates to novel hydrolytically stable carbocyclic nucleoside analogues of 5-aza-2′-deoxycytidine and 5-aza-cytidine and their use as hypomethylating agents.

5-Aza-2′-deoxycytidine (decitabine, AzadC) and 5-aza-cytidine (vidaza, AzaC) are nucleoside analogues that act as an antimetabolite by manipulating epigenetic information [1-5]. Epigenetic information in DNA is associated with the formation of 5-methyl-2′-deoxycytidine (mdC) from 2′-deoxycytidine (dC) with the help of DNA methyltransferases (DNMTs) and S-adenosylmethionine (SAM) as the methylating cofactor [6-7, 4]. Methylation of dC to mdC in promoter regions is typically associated with transcriptional silencing of genes [8-9]. AzadC is a prodrug that is inside cells converted into the corresponding active triphosphate and subsequently incorporated into the genome during cell division. AzaC is a prodrug as well, that is 2′-deoxygenated and subsequently converted into the corresponding 5′-triphosphate for incorporation into DNA. AzaC is also converted into the 5′-triphosphate without prior 2′-deoxygenation and incorporated into RNA. The mode of action of Aza(d)C involves reaction of its electrophilic C6 positions with a DNMT active site thiol nucleophile as shown in FIG. 1a [10-11]. This generates a covalent intermediate that is methylated by the SAM cofactor, hold in close proximity. Due to the N-atom at position 5 of the triazine heterocycle, the final beta-elimination reaction, which would usually release m(d)C from the DNMT enzyme is not possible anymore. The consequence is the formation of a covalent DNA-DNMT or RNA-DNMT crosslink.

As a result of administering Aza(d)C, a large drop of mdC levels, i.e. a hypomethylating effect is observed, which leads to the reactivation of silenced tumor suppressor genes in cancer cells [1], which allows re-differentiating cancer cells back into normally proliferating cells or gives rise to cell death. Aza(d)C is currently in use as a medicament for the treatment of myelodysplastic syndromes (MDS) [2] and acute myeloid leukemia (AML) [4]. Clinically, it is administered in several cycles, with each cycle involving one week of treatment and three weeks of pausing.

A problem associated with Aza(d)C is that it hydrolyses in aqueous solution following the path depicted in FIG. 1b. This hydrolysis compromises the activity of Aza(d)C, particularly over the long treatment times.

The present inventors have overcome this problem by providing analogues of Aza(d)C that can demethylate (and hence react with an S-nucleophile), while hydrolysis (reaction with an O-nucleophile) is blocked. Surprisingly, they have found that replacing the oxygen of the deoxyribose by a CH₂-group has a large remote effect on the reactivity of the heterocycle. These analogues, particularly a carbocyclic version of Aza(d)C still inhibit methylation by DNA methyltransferases but are hydrolytically stable as shown in FIG. 1c.

A first aspect of the present invention relates to a compound of Formula I:

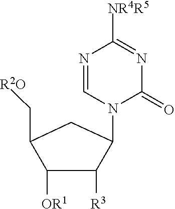

wherein R¹ is H, a free or protected, modified or unmodified phosphate group, or a hydroxyl-protecting group,
R² is H, a free or protected, modified or unmodified phosphate group, or a hydroxyl-protecting group,
R³ is H, F, CH₃, CH₂F, CHF₂, CF₃, OH or OR⁶, wherein R⁶ is a hydroxyl-protecting group, and
R⁴ and R⁵ are H or form an amino-protecting group,
or a salt thereof.

In the compounds of Formula I, R¹ may be selected from H, a free or protected, modified or unmodified phosphate group, e.g. a monophosphate, diphosphate or triphosphate group, or a hydroxyl-protecting group.

The term “phosphate group” includes an unmodified phosphate group, e.g. an unmodified monophosphate, unmodified diphosphate or unmodified triphosphate group or a modified phosphate group, e.g. a modified monophosphate, modified diphosphate or modified triphosphate group, wherein one or more oxygen atoms are replaced by carbon, sulfur and/or nitrogen atoms. Examples of modified phosphate groups are phosphonate, phosphoramidate or phosphorothioate groups. The term “phosphate group” also includes free phosphate groups and protected phosphate groups, e.g. phosphate esters including monoesters and diesters, and amidates including monoamidates, diamidates and amidate esters. Detailed reviews of protected phosphate and phosphonate groups are found in [28] and [29], the contents of which are herein incorporated by reference. Thus, in pharmaceutical applications, R¹ may be a group that is a protected phosphate, diphosphate or triphosphate group to liberate a phosphate, diphosphate or triphosphate group upon cleavage in the cell (prodrug concept).

Hydroxyl-protecting groups are known in the art, particularly in the field of nucleoside chemistry and include, but are not limited to, acyl groups such as acetyl or benzoyl, benzyl groups, trityl groups such as trityl, methoxytrityl or dimethoxytrityl, silyl groups such as trimethylsilyl, t-butyl-dimethylsilyl or tri-i-propysilyl, or alkyl- or substituted alkyl groups such as methyl, ethoxyethyl or beta-methoxyethoxym ethyl.

R² may be selected from H, an unmodified or modified, free or protected phosphate group as described above, e.g. a monophosphate, diphosphate or triphosphate group, or a hydroxyl-protecting group as described above. Thus, in pharmaceutical applications, R² may be a group that is a protected phosphate diphosphate or triphosphate group to liberate a phosphate, diphosphate or triphosphate group upon cleavage in the cell (prodrug concept).

In certain embodiments, R³ may be H. In such a case, the 5-membered carbocyclic ring in Formula I can be considered as a deoxyribose wherein the oxygen atom at positon 1′ of the ring has been replaced by a CH₂-group. In further embodiments, R³ may be OH or OR⁶, wherein R⁶ is a hydroxyl-protecting group as described above. In these embodiments, the 5-membered carbocyclic ring in Formula I may be considered as a ribose wherein the oxygen atom at positon 1′ of the ring has been replaced by a CH₂-group. R³ may also be CH₃, CH₂F, CHF₂, CF₃ or F In these embodiments, the 5-membered carbocyclic ring in Formula I may be considered as a ribose analogue wherein the oxygen atom at positon 1′ of the ring has been replaced by a CH₂-group.

R⁴ and R⁵ each may be H, thus providing a primary amino group NH₂ or they may form an amino-protecting group, e.g. wherein one or both H-atoms are replaced by the protecting group. Amino-protecting groups are known in the art, particularly in the field of nucleoside chemistry and include, but are not limited to, acyl groups such as acetyl or benzoyl, benzyl groups, carbobenzyloxy groups, tosyl groups, 9-fluorenyl methyloxycarbonyl groups or t-butyloxycarbonyl groups.

In certain embodiments, R¹ is H.

In certain embodiments, R² is H or an unmodified or modified phosphate group, e.g. a monophosphate, diphosphate or triphosphate group including a free or protected monophosphate, diphosphate or triphosphate group.

In certain embodiments, R³ is H or OH.

In certain particular embodiments R¹, R², R³, R⁴ and R⁵ each are H, or R¹, R², R⁴, and R⁵ each are H and R³ is OH or OR⁶. In an especially particular embodiment, R¹, R², R³, R⁴ and R⁵ are H. This compound is a carbocyclic analogue of AzadC (decitabine) which—as indicated above—has a surprisingly high hydrolytic stability while maintaining inhibitory activity towards methyltransferases, particularly DNA methyltransferases. In a further especially particular embodiment, R¹, R², R⁴ and R⁵ are H and R³ is OH. This compound is a carbocyclic analogue of AzaC (vidaza).

In further particular embodiments, the invention also relates to prodrugs of AzadC and AzaC, particularly to compounds, where R¹ and/or R² are different from H.

The present invention also encompasses salts of the compounds of Formula I, in particular pharmaceutically acceptable salts. The compounds of the present invention are capable of forming acid and/or base salts by means of the presence of amino and/or phosphate groups or groups similar thereto. Acid addition salts may be formed with inorganic acids and organic acids and include, but are not limited to, acetate, benzoate, hydrobromide, hydrochloride, citrate, lactate, phosphate, tosylate and trifluoroacetate salts. Base addition salts can be formed within inorganic bases and organic bases and include, but are not limited to, ammonium, sodium, potassium, calcium, magnesium salts or salts derived from primary, secondary and tertiary amines.

The compounds of the present invention act as antimetabolites by reducing the level of the epigenetic modification 5-methyl-2′-deoxycitidine (mdC). They may be incorporated into the genome and/or the RNA of proliferating cells and inhibit DNA- and/or RNA-methyltransferases leading to a reduction of mdC in DNA and mC in RNA. The loss of mdC in DNA, which is a transcriptional silencer in promoters, leads to the reactivation of genes including tumor suppressor genes, which elicits an anti-hyperproliferative effect.

Thus, a further aspect of the further invention relates to a compound of Formula I as described above or a pharmaceutically acceptable salt thereof for the use in medicine, e.g. in veterinary or human medicine.

In certain embodiments, the compound of the present invention is used for the treatment of a hyperproliferative disorder including malignant or non-malignant hyperproliferative disorders. The term “hyperproliferative disorder” relates to disorders caused by, associated with and/or accompanied by a dysfunctionally increased proliferation of cells, tissues and/or organs within an organism.

Thus, the invention also relates to a method of treating a hyperproliferative disorder, comprising administering to a subject in need thereof, particularly to a human subject, a therapeutically effective dose of a compound of Formula I or a pharmaceutically acceptable salt thereof.

In particular embodiments, the compounds of the present invention are used for the treatment of cancer. In certain embodiments, the compounds of the present invention are used for the treatment of leukemia, e.g. acute myeloid leukemia (AML) or a myelodysplastic syndrome (MDS) including previously treated or untreated, primary or in secondary MDS such as refractory anemia, refractory anemia with ringed sideroblasts, refractory anemia with excess blasts, refractory anemia with excess blasts in transformation and chronic myelomonocytic leukemia as well as intermediate-1, intermediate-2 and high risk MDS.

In further embodiments, the compounds of the invention may be used for the treatment of renal insufficiency based on results reported for decitabine [26]. In certain embodiments, the compounds of the present invention may be used for the treatment of atherosclerosis based on the observation that decitabine prevents atherosclerosis lesion formation and reduces the production of inflammatory cytokines by macrophages [27].

A compound of Formula I or a pharmaceutically acceptable salt thereof may be administered to a subject in need thereof as a pharmaceutically composition comprising the active agent and a pharmacologically acceptable carrier. The composition may be administered in any suitable way, e.g. orally, parenterally, by inhalation and/or by dermal application. The carrier may be any suitable pharmaceutical carrier. The compound of Formula I is administered in a therapeutically effective dose which may be determined by the skilled practitioner based on the subject and the disorder to be treated.

In pharmaceutical applications, the composition may be in the form of a solid dosage form, e.g. a tablet, a capsule etc. or a liquid dosage form, e.g. a solution, emulsion, suspension or the like.

A compound of Formula I may be administered in one or several treatment cycles, wherein each treatment cycle may involve at least 1, 2, 3, 4, 5, 6, 7 or 10 days.

A compound of Formula I may be administered as a monotherapy or in is combination with a further medicament, particularly a medicament suitable for the treatment of hyperproliferative disorders as described above. In certain embodiments a compound of Formula I may be administered as a combination therapy together with an anti-cancer drug different from Formula I.

Further, the compounds of Formula I are also useful for non-medical applications, e.g. generally in the fields of basic research, diagnostics and/or drug screening. Thus, in certain embodiments, the invention relates to an in vitro use of a compound of Formula I for inhibiting the methylation of nucleic acids including DNA and RNA, particularly for inhibiting the methylation of DNA, more particularly for inhibiting the methylation of genomic DNA. Thus, the compounds are useful for a genome analysis in cells, e.g. eukaryotic cells such as animal cells including mammalian and human cells, particularly for an epigenetic analysis.

A general scheme for the preparation of the compounds of Formula I is depicted in the following scheme. The synthesis starts with a t-butyloxycarbonyl (Boc)-protected aminocyclopentane derivative 2, which has previously been used to synthesize DNA lesion analogues [12-15]. Compound 2 was first benzyl-protected to 3, Boc-deprotected to 4, and then reacted with carbimidazole 5, which was prepared in two steps from isomethylurea 6 after generation of the free base 7 with potassium hydroxide and reaction of 7 with carbonyldiimidazole. This provides the carbamoylurea-cyclopentane nucleoside analogue 8. Cyclization to the triazine base 9 was subsequently performed with triethylorthoformate. Reaction of 9 with NH₃ in methanol and deprotection of the benzyl groups with BCl₃ in dichloromethane furnished the final compound cAzadC 1 as the free nucleoside.

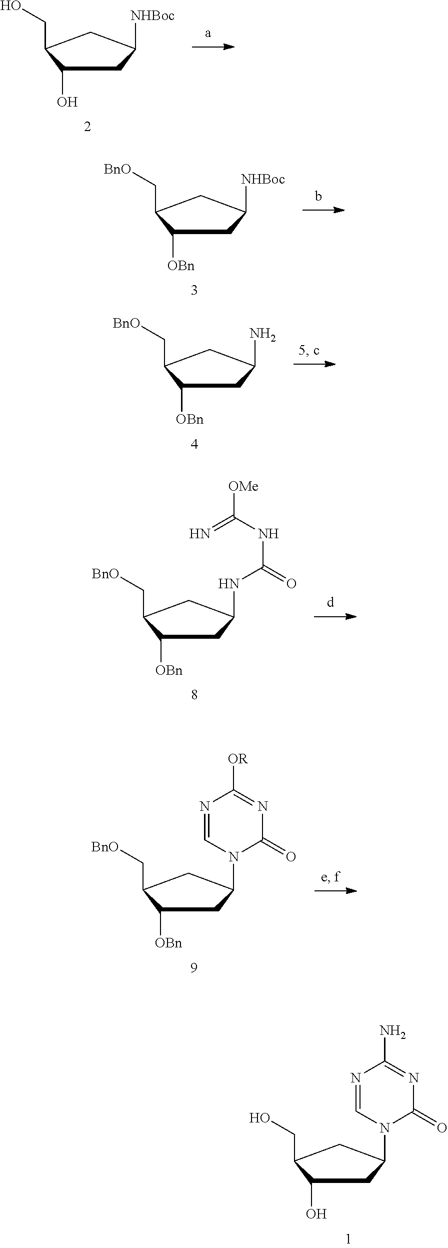

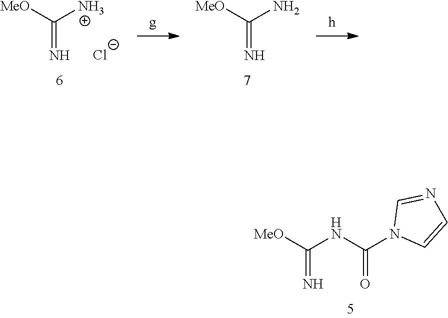 [SMILES: COC(=N)[NH3+] ; [Cl-] ; COC(=N)N ; COC(=N)NC(=O)n1ccnc1]

Recrystallization of compound cAzadC 1 from hot methanol gave colourless needles, which allowed us to solve the crystal structure that is depicted in FIG. 2. Interesting is the observation that cAzadC 1 exists with two different cyclopentane conformations in the crystal. One of these conformations is typical for 2′-deoxynucleosides in DNA showing that the cAzadC 1 nucleoside can adopt the is correct DNA-type conformation and has the potential to become phosphorylated and integrated into the genome.

Another general scheme for the preparation of the compounds of Formula I is depicted in the following showing the synthesis of the carbacyclic version of azacitidine (vidaza) cAzaC, 20:

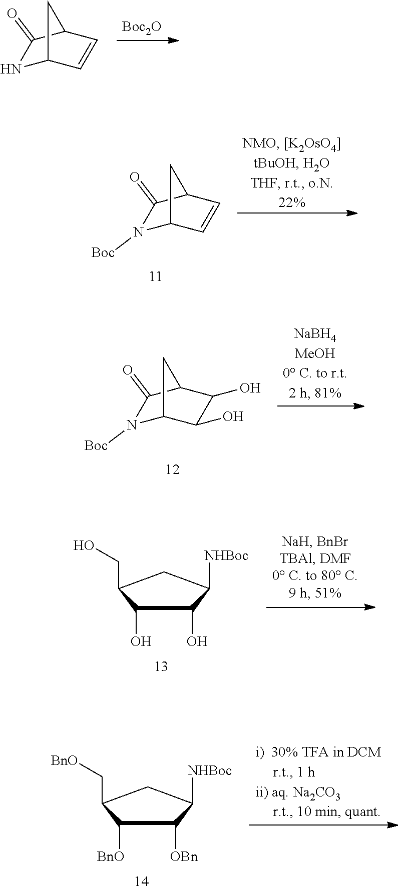

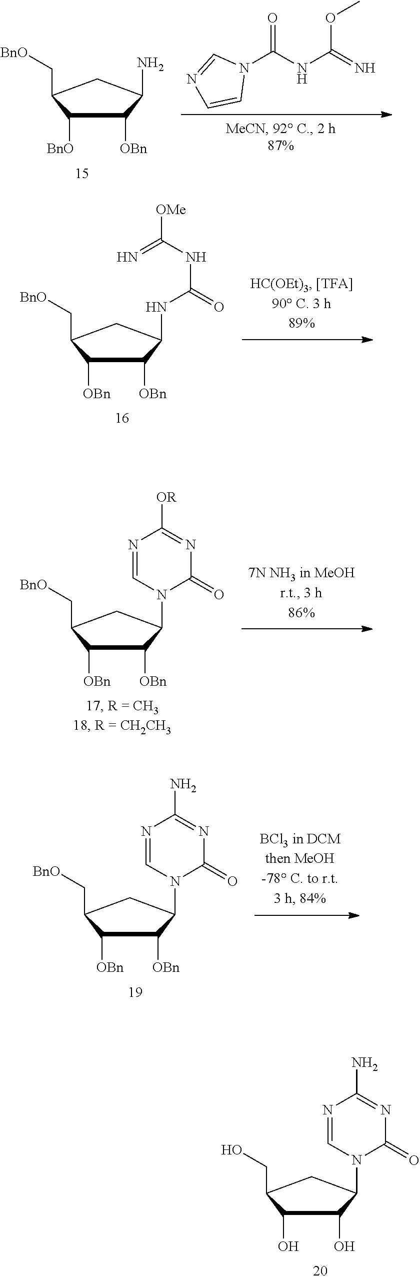

A general scheme for the preparation of prodrugs of the compounds of Formula I is depicted in the following showing the synthesis of a 5′ phoshoroamidate prodrug 24 of cAzaC, 20:

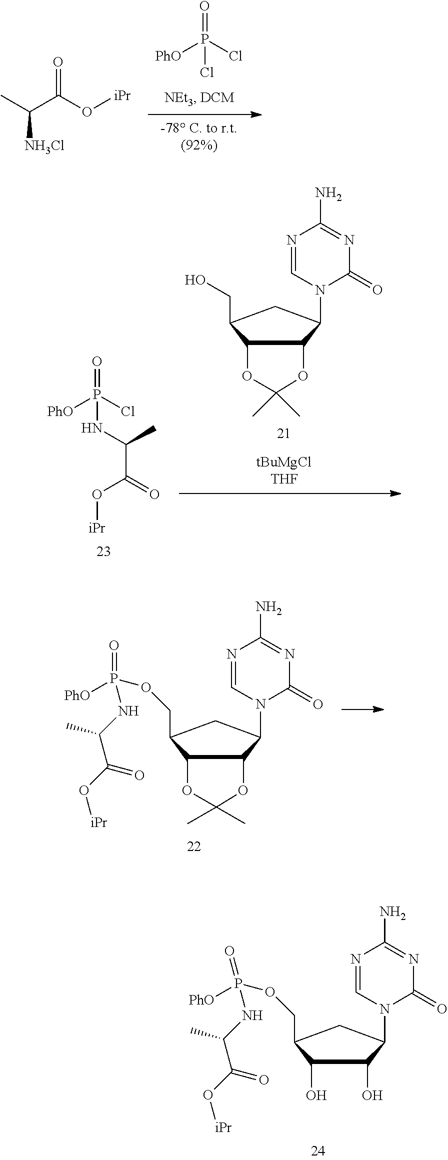

Further, the present invention shall be explained in more detailed by the following Figures and Examples.

### FIGURE LEGENDS

FIG. 1. Depiction of 5-aza-2′-deoxycytidine (decitabine, AzadC) together with its mode of action. a) Active site thiol reacts with the C6-position of AzadC. b) Hydrolytic degradation pathway that goes in hand with reaction of a water molecule with the C6-position (O-reactivity) of AzadC. This leads to a final base loss and formation of an abasic site. c) Boxed, depiction of the carbocyclic version cAzadC 1.

FIG. 2. Crystal structure of carbocyclic 5-aza-2′-deoxycytidine (cAzadC 1) showing the molecule in the observed C6′-endo conformation (a) and the C2′-endo conformation (2T1) (b).

FIG. 3. HPLC-based stability measurements showing (a) severe hydrolytic decomposition of 5-aza-2′-deoxycytidine (AzadC) solutions at different pH values, while (b) the carbocyclic compound cAzadC 1 was stable at all three pH values. The inset table in (a) shows the chromatogram between t1=10 min and t2=20 min for AzadC. The AzadC signal is depicted in red.

FIG. 4. Depiction of the quantification data of DNA modifications of carbocyclic 5-aza-2′-deoxycytidine-treated (cAzadC 1) mouse embryonic stem cells (mESC) obtained by UHPLC-MS2. For each condition, three biological replicates were measured in technical triplicates. For each technical replicate, 0.5 μg of DNA were digested. Bar graphs represent mean, error bars represent standard deviation. LLOQ indicates the lower limit of quantification.

FIG. 5. HPLC investigation of a neutral solution of Decitabine 1 (left) versus the carbacyclic compound cAzaC 20 (right). 100 mM solutions of the compounds were kept in pure water and tested at t=0 h, 1.5 h, 3 h, 4.5 h, 6 h, 24 h, 6 d.

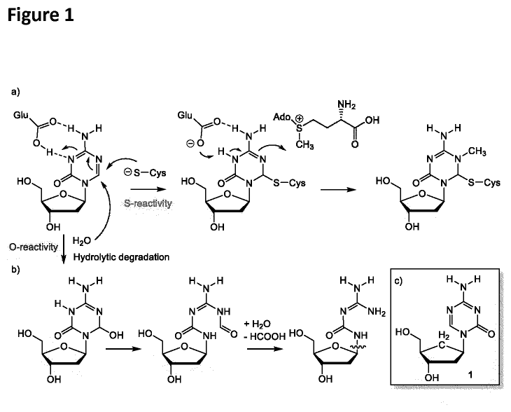

### EXAMPLES

### Example 1—Synthetic Procedures for cAzadC (1)

### Tert.-Butyl-N-[(1R,3S,4R)-3-hydroxy-4-(hydroxymethyl)cyclopentyl]-carbamate (2)

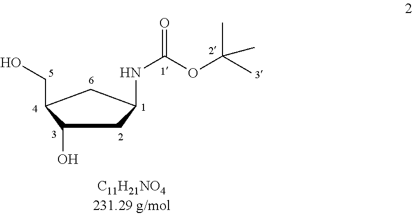 [SMILES: CC(C)(C)OC(=O)N[C@@H]1C[C@H](CO)[C@H](O)C1]

Compound 2 was synthesized from tert-Butyl-N-((1S,2R,4R,5R)-4-((tert-butyl -dimethylsilyloxy)methyl)-6-oxa-bicyclo-[3.1.0]-hex-2-yl)-carbamate as described previously [1].

### Tert.-Butyl-N-[(1R,3S,4R)-3-benzyloxy-4-(benzyloxymethyl)-cyclopentyl]-carbamate (3)

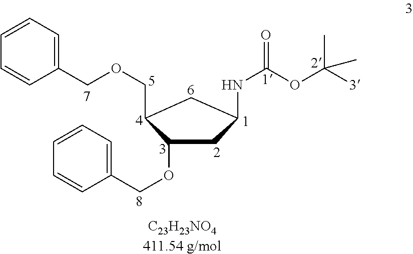

Boc-protected 2 (3.107 g, 13.43 mmol, 1.0 eq.) was dissolved in dry DMF (75 mL). The solution was cooled to 0° C. and NaH (60% dispersion in mineral oil, 1.182 g, 29.55 mmol, 2.2 eq.) was added in three portions. After 15 min at 0° C., benzyl bromide (4.00 mL, 33.58 mmol, 2.5 eq.) was added dropwise. The reaction mixture was stirred for 1.5 h at 0° C. and for additional 2 h at rt. The reaction mixture was diluted using CH₂Cl₂ (30 mL) and the reaction was quenched with sat. is NaHCO₃ (30 mL). The mixture was extracted with CH₂Cl₂ (500 mL). The organic phase was washed three times with sat. NaHCO₃ (1×400 mL, 2×100 mL) and dried over Na₂SO₄. The solvent was removed in vacuo. The crude product was purified by column chromatography on silica gel with a stepwise gradient iHex/EtOAc (9:1→7:1→4:1) to afford product 3 as a colorless solid (3.39 g, 8.24 mmol, 61%).

R_{f}=0.67 (iHex/EtOAc=2:1)

Mp=74-76° C.

HRMS [ESI+]: [C₂₅H₃₃NO₄Na]⁺, [M+Na⁺]⁺ calculated.: 434.2292, found: 434.2299

¹H-NMR (599 MHz, CDCl₃): 5/ppm=7.39-7.26 (m, 10H, Ar—H), 4.76 (s, 1H, NH), 4.53-4.37 (m, 4H, C₇—H and C₈—H), 4.18-4.08 (m, 1H, C₁—H), 3.96-3.87 (m, 1H, C₃—H), 3.53-3.41 (m, 2H, C₅—H), 2.36-2.25 (m, 2H, C₆—Hₐ, C₄—H), 2.11-2.05 (m, 1H, C₂—Hₐ), 1.77-1.68 (m, 1H, C₂—H_{b}), 1.43 (s, 9H, C_{2′}—H), 1.29-1.20 (m, 1 H₇ C₆—H_{b}).

¹³C-NMR (151 MHz, CDCl₃): δ/ppm=155.5 (C_{1′}), 138.60, 138.23, 128.36, 128.28, 127.59, 127.56, 127.42 (C—Ar), 81.0 (C₃), 79.2 (C_{2′}), 73.3 (C₈), 71.9 (C₅), 71.3 (C₇), 50.4 (C₁), 44.9 (C₄), 39.6 (C₂), 35.0 (C₆), 28.6 (C_{2′}).

FTIR (ATR):=\tilde{ν}/cm ⁻¹3339 (w), 2973 (w), 2929 (w), 2858 (w), 1692 (m), 1496 (m), 1453 (m), 1390 (w), 1364 (m), 1274 (m), 1247 (m), 1166 (s), 1091 (s), 1067 (s), 1027 (m), 1012 (m).

### (1R, 3S, 4R)-1-Amino-3-benzyloxy-4-(benzyloxymethyl)cyclopentane (4)

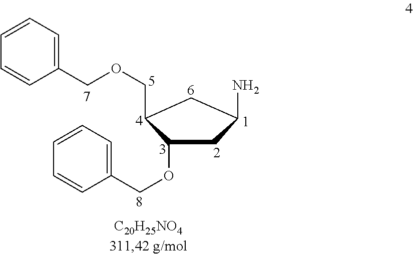

A solution of Boc-protected amine 3 (3.351 g, 8.14 mmol, 1.0 eq.) and trifluoroacetic acid (12 mL, 30% (v/v)) in dry CH₂Cl₂ (28 mL) was stirred for 1 h at rt. After removing the solvent in vacuo, the residue was resuspended in sat. Na₂CO₃ (100 mL) and stirred for 10 min. The mixture was extracted with EtOAc (3×150 mL) and the combined organic phases were dried over Na₂SO₄. Removing the solvent in vacuo resulted in the free amine 4 as viscous brown oil, which was used without further purification due to its instability towards O₂.

Analytical data of purified 4:

HRMS [ESI⁺]: [C₂₃H₂₉N₄O₄Na]⁺, [M+Na⁺]⁺ calculated: 312.1958, found: 312.1955

¹_{H-NMR} (400 MHz, CDCl₃): δ/ppm=7.38-7.20 (m, 10H, C—Ar), 4.57-4.36 (m, 4H, C₇, C₈), 3.95-3.85 (m, 1H, C₁), 3.62-3.50 (m, 1H, C₃), 3.50-3.39 (m, 2H, C₅), 2.41-2.28 (m, 1H, C₄), 2.28-2.13 (m, 1H, C₆—Hₐ), 2.13-1.96 (m, 3H, NH₂, C₂—Hₐ), 1.65-1.50 (m, 1H, C₂—H_{b}), 1.19-1.08 (m, 1H, C₆—Hₐ),

¹³C-NMR (101 MHz, CDCl₃): δ/ppm=138.8, 138.4, 128.5, 128.5, 127.79, 127.75, 127.7, 127.6 (C—Ar), 81.82 (C₃), 73.23 (C₇), 72.58 (C₅), 71.10 (Cₛ), 51.40 (C₁), 45.63 (C₄), 42.07 (C₂), 38.08 (C₆).

### 2-Methyl-1-(1 -imidazoylcarbonyl)-isourea (5)

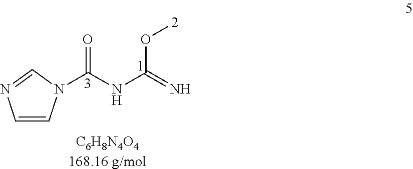

To a solution of O-methylisourea hydrochloride 6 (5.00 g, 45.65 mmol, 1.0 eq.) in Et₂O:H₂O (39:1) at −15° C., KOH powder (51.22 g, 912.96 mmol, 20.0 eq.) was added portion wise under constant stirring. After 30 min, the mixture was filtrated, and the residue was washed with ice-cold Et₂O (3×50 mL). The combined organic phases were concentrated to 20 mL in vacuo. Cooling to −20° C. resulted in precipitation. Vacuum filtration under N₂ flow resulted in O-methylisourea 7 as a colorless wax (1.759 g, 23.74 mmol, 52%). A solution of 7 (1.24 g, 16.74 mmol, 1.0 eq.) and carbonyldiimidazole (2.90 g, 17.91 mmol, 1.07 eq.) in dry THF (35 mL) was stirred for 3 h at rt. After 1 h, a colorless precipitate was observed. The reaction mixture was concentrated to 3 mL in vacuo and filtrated. The residue was washed with ice-cold THF (2×3 mL) and the combined organic phases were lyophilized, which resulted in colorless solid 5 (1.946 g, 11.57 mmol, 69%).

¹H-NMR (400 MHz, CDCl₃): δ/ppm=8.60 (br s, 1H, N—H), 8.35 (s, 1H, Ar—H), 7.58 (s, 1H, Ar—H), 7.04 (s, 1H, Ar—H), 6.04 (br s, 1H, N—H), 3.96 (s, 3H, C₂—H).

¹³C-NMR (101 MHz, CDCl₃): δ/ppm=165.7 (C₁), 157.3 (C3), 137.5(Ar), 130.0 (Ar), 117.2 (Ar), 55.3 (C₂).

### 1-[(1′R,3′S,4′R)-3′-Benzyloxy-4′-(benzyloxymethyl)-cyclopentyl]-methylisobiuret (8)

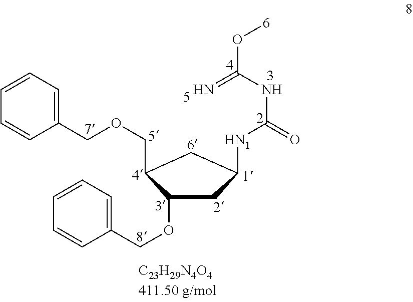

The crude product 4 was dissolved in dry acetonitrile (75 mL) and the carbonyl imidazole 5 (1.506 g, 8.96 mmol, 1.1 eq.) was added. The mixture was refluxed for 2 h. The solvent was removed in vacuo. The crude product was purified by column chromatography on silica gel with a stepwise gradient of iHex/EtoAc (2:1→1:1) to obtain the methylisobiurete 8 as a yellow oil (2.613 g, 6.35 mmol, 78%).

R_{f}=0.51 (DCM/MeOH=19:1)

HRMS [ESI⁺]: [C₂₃H₂₉N₄O₄Na]⁺, [M+Na⁺]⁺ calculated: 434.2050, found: 434.2049

¹H-NMR (599 MHz, CDCl₃): δ/ppm=7.35-7.16 (m, 10H, Ar—H), 5.42 (d,³J_{1′,N1} =7.8 Hz, 1H, N₁—H), 4.50-4.34 (m, 4H, C_{7′}—H_{a,b}, C_{8′}—H_{a,b}), 4.33-4.16 (m, 1H, C_{1′}—H), 3.93-3.81 (m, 1H, C₃—H), 3.62 (s, 3H, C₆—H), 3.43-3.31 (m, 2H,C_{5′}—H), 2.34-2.18 (m, 2H, C_{6′}—Hₐ, C_{4′}—H), 2.06 (ddd, ²J_{2′a,2′b}=13.0 Hz, ³J_{2′a,1′/3′}=6.9 Hz, ³J_{2′a,1′/3′}=4.6 Hz, 1H, C_{2′}—Hₐ), 1.73 (ddd, ²J_{2′a,2b′}=13.0 Hz, ³J_{2′b,1′/3′}=7.0 Hz, ³J_{2′b,1′/4′}=7.0 Hz, 1H, C₂—H_{b}), 1.28-1.11 (m, 1H,C_{6′}—H_{b})

¹³C-NMR (151 MHz, CDCl₃): δ/ppm=163.8 (C₂), 162.3 (C₄), 138.9 (C_{Ar}), 138.5 (C_{Ar}), 128.51 (C_{Ar}), 128.41 (C_{Ar}), 127.76 (C_{Ar}), 127.74 (C_{Ar}), 127.70 (C_{Ar}), 127.56 (C_{Ar}), 81.1 (C_{3′}), 73.3 (C_{7′}), 71.9 (C_{5′}), 71.2 (C_{8′}), 53.8 (C₆), 49.5 (C_{1′}), 45.0 (C_{4′}), 39.5 (C_{2′}), 35.0 (C_{6′}).

### 4-Ethoxy-1-[(1′R,3′S,4′R)-3′-benzyloxy-4′-(benzyloxymethyl)-cyclopentyl]-1H -[1,3,5]-triazin-2-one (9a) and 4-methoxy-1-[(1′R,3′S,4′R)-3′-benzyloxy-4′-(benzyloxymethyl)-cyclopentyl]-1H-[1,3,5]-triazin-2-one (9b)

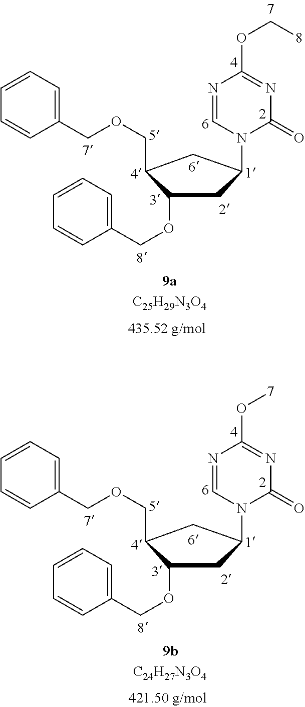

Trifluoroacetic acid (75 μL) was added to a solution of 8 (2.613 g, 6.35 mmol, 1.0 eq) in triethyl orthoformate (60 mL). The mixture was refluxed for 3 h. The solvent was removed in vacuo. The residue was co-evaporated once with MeOH and the crude product was purified by column chromatography on silica gel with a stepwise gradient of iHex/EtOAc (2:1→1:1→1:2) to obtain 9a (1.774 g, 4.07 mmol, 64%, colorless solid) and 9b (268.0 mg, 0,64 mmol, 10%, colorless oil).

9a:

R_{f}=0.71 (iHex/EtOAc, 1:3)

HRMS [ESI⁺]: [C₂₅H₃₀N₃O₄H]⁺, [M+H⁺]⁺ calculated: 436.2232, found.: 436.2231.

¹H-NMR (400 MHz, CDCl₃): δ/ppm=8.23 (s, 1H, C₆—H), 7.38-7.24 (m, 10H, Ar—H), 5.07-4.89 (m, 1H, C_{1′}—H), 4.56-4.39 (m, 6H, C_{7′}—H_{a,b}, C_{8′}—H_{a,b}, C₇—H_{a,b}), 4.07-3.08 (m, 1H, C_{3′}—H), 3.61-3.46 (m, 2H, C_{5′}—H_{a,b}), 2.52-2.36 (m, 2H, C_{6′}—Hₐ, C₄—H), 2.28 (ddd, 1H, ²J_{2′a,2′b}=13.3 Hz, ³J=7.6 Hz, ³J=2.7 Hz, C_{2′}—Hₐ), 2.11 (ddd, 1H, ²J_{2′a,2′b}=13.3 Hz, ³J=10.0 Hz, ³J=6.3 Hz, C_{2′}—H_{b}), 1.84-1.68 (m, 1H, C_{6′}—H_{b}), 1.40 (t, 3H, ³J_{8, 7}=7.1 Hz, C₈—H),

¹³C-NMR (101 MHz, CDCl₃): δ/ppm=169.3 (C₄), 158.4 (C₆), 155.0 (C₂), 138.2 (C_{Ar}), 138.1 (C_{Ar}), 128.6 (C_{Ar}), 128.6 (C_{Ar}), 127.93 (C_{Ar}), 127.85 (C_{Ar}), 127.82 (C_{Ar}), 80.4 (C_{3′}), 73.4 (C_{7′/8′}), 71.26 (C_{7′/8′}), 71.25 (C_{5′}), 65.1 (C₇), 56.8 (C_{1′}), 44.9 (C_{4′}), 36.9 (C_{2′}), 32.6 (C_{6′}), 14.2 (C₈).

9b:

R_{f}=0.63 (iHex/EtOAc, 1:3)

HRMS [ESI⁺]: [C₂₄H₂₈N₃O₄]⁺, [M+H⁺]⁺ calculated: 422.2074 , found: 422.2075.

¹H-NMR (599 MHz, CDCl₃): δ/ppm==8.23 (s, 1H, C₆—H), 7.38-7.25 (m, 10H, Ar—H), 5.04-4.93 (m, 1H, C_{1′}—H), 4.57-4.43 (m, 6H, C_{7′}—H_{a,b}, C_{8′}—H_{a,b}), 4.09-3.98 (m, 4H, C_{3′}—H, C₇—H), 3.60-3.50 (m, 2H, C_{5′}—H_{a, b}), 2.50-2.39 (m, 2H, C_{6′}—Hₐ, C_{4′}—H), 2.28 (ddd, 1H, ²J_{2′a,2′b}=13.1 Hz, ³J=7.4 Hz, ³J=2.7 Hz, C_{2′}—Hₐ), 2.11 (ddd, 1H, ²J_{2′a,2′b}=13.1 Hz, ³J=10.1 Hz, ³J=6.3 Hz, C_{2′}—H_{b}), 1.80-1.68 (m, 1H, C_{′6}—H_{b}).

¹³C-NMR (101 MHz, CDCl₃): δ/ppm=169.9 (C₄), 158.5 (C₆), 155.0 (C₂), 138.3 (C_{Ar}), 138.0 (C_{Ar}), 128.63 (C_{Ar}), 128.57 (C_{Ar}), 127.93 (C_{Ar}), 127.84 (C_{Ar}), 127.82 (C_{Ar}), 80.4 (C_{3′}), 73.5 (C_{7′/8′}), 71.3 (C_{7′/8′}), 56.9 (C_{1′}), 56.0 (C₇), 44.9 (C_{4′}), 36.9 (C_{2′}), 32.6 (C_{6′}).

### 4-Amino-1 -[(1′R,3′S,4′R)-3′-benzyloxy-4′-(benzyloxymethyl)-cyclopentyl]-1H -[1,3,5]triazin-2-one (10)

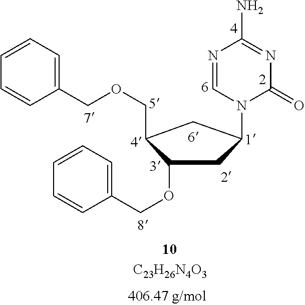

Ethoxytriazine 9a (1.630 g, 3.74 mmol, 1.0 eq.) was dissolved in methanolic NH₃ (7 N in MeOH, 60 mL) and stirred for 3 h at rt. The mixture was diluted with H₂O (340 mL), resulting in a colorless precipitate, which was extracted with EtOAc (3×200 mL). The organic phases were combined, dried and the solvent was removed in vacuo, which is resulted in the benzyl-protected cAzadC 10 (1.478 g, 3.63 mmol, 97%) as a colorless foam. Synthesis of 10 was also successful starting from the methoxytriazine 9b and following the same procedure.

R_{f}=0.50 (CH₂Cl₂/MeOH, 9:1)

HRMS [ESI⁺]: [C₂₃H₂₇N₄O₃]⁺, [M+H⁺] calculated: 407.2078, found: 407.2081.

¹H-NMR (599 MHz, CDCl₃): δ/ppm=8.01 (s, H, C₆—H), 7.35-7.23 (m, 10H, Ar—H), 5.78 (5, 2H, NH), 4.92-4.76 (m, 4H, C_{7′}—H and C_{8′}—H), 4.59-4.46 (m, 1H, C₁′—H), 4.07-3.95 (m, 1H, C_{3′}—H), 3.55-3.45 (m, 2H, C_{5′}—H), 2.44-2.28 (m, 2H, C_{6′}—Hₐ, C_{4′}—H), 2.24-2.16 (m, 1H, C_{2′}—Hₐ), 2.14-2.05 (m, 1H, C_{2′}—H_{b}), 1.78-1.67 (m, 1H, C_{6′}—H_{b})

¹³C-NMR (151 MHz, CDCl₃): δ/ppm=165.7 (C₄), 157.1 (C₆), 154.2 (C₂), 138.3, 138.3, 128.6, 128.5, 127.84, 127.78 (C—Ar), 80.4 (C_{3′}), 73.4 (C_{8′}), 71.4 (C_{5′}), 71.2 (C_{8′}), 56.7 (C_{1′}), 44.9 (C_{4′}), 36.8 (C_{2′}), 32.5 (C_{6′}).

### 4-Amino-1-[(1′R,3′S,4′R)-3′-hydroxy-4′-(hydroxymethyl)-cyclopentyl]-1H -[1,3,5]triazin-2-one (cAzadC, 1)

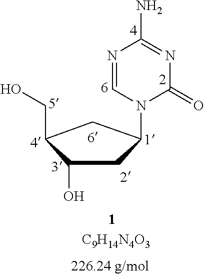

A solution of benzyl-protected 10 (1.478 g, 3.63 mmol, 1.0 eq) in CH₂Cl₂ (100 mL) was cooled to −78° C. and BCl₃ (1 M in DCM, 12.7 mL, 12.7 mmol, 3.5 eq.) was added dropwise. The mixture was stirred for 1 h at −78° C. and stirred for additional 2 h at rt. The reaction was quenched by addition of MeOH (85 mL) under constant stirring. The solvent was removed in vacuo. The crude product was purified by column chromatography on silica gel with a stepwise gradient CH₂Cl₂/MeOH (9:1→7:3) to obtain cAzadC (1, 0.740 g, 3.27 mmol, 90%). Recrystallization from hot MeOH resulted in 57% cAzadC (1, 465.3 mg, 2.06 mmol) as colorless acicular monocrystals.

R_{f}=0.1 (CH₂Cl₂/MeOH, 9:1)

Mp.: 198-200° C.

HRMS [ESI⁺]: [C₉H₁₅N₄O₃]⁺, [M+H⁺]⁺ calculated: 227,1138, found: 227,1139.

¹H-NMR (400 MHz, D₂O): δ/ppm=8.29 (s, 1H, C₆—H), 4.90-4.62 (m, 1H, C_{1′}—H, Overlap with D₂O signal), 4.29-4.16 (m, 1H, C_{3′}—H), 3.73 (dd, ⁵J_{5′a,5′b}=11.2 Hz, ³J_{5′a,4′}=5.7 Hz, 1H, C_{5′a}—H ), 3.62 (dd, ⁵J_{5′a,5′b}=11.2 Hz, ³J_{5′b,4′}=6.8 Hz, 1H, C_{5′b}—H), 2.41-2.29 (m, 1H, C_{6′a}—H), 2.28-2.18 (m, 1H, C_{2′a}—H), 2.17-2.02 (m, 2H, C_{2′, b}—H, C_{4′b}—H), 1.77-1.55 (m, 1H, C_{6′b}—H).

¹³C-NMR (101 MHz, CDCl₃): δ/ppm=165.5 (C₄), 158.5(C₆), 156.4(C₂), 72.2 (C_{3′}), 62.8 (C_{5′}), 56.2 (C_{1′}), 48.11 (C_{4′}), 38.0 (C_{2′}), 32.0 (C_{6′}.).

FTIR (ATR): \tilde{ν}/cm ⁻=3194 (m), 1653 (m), 1540 (s), 1437 (s), 1278 (s), 1036 (s).

### Example 2—X-Ray Crystal Structure Analysis of cAzadC

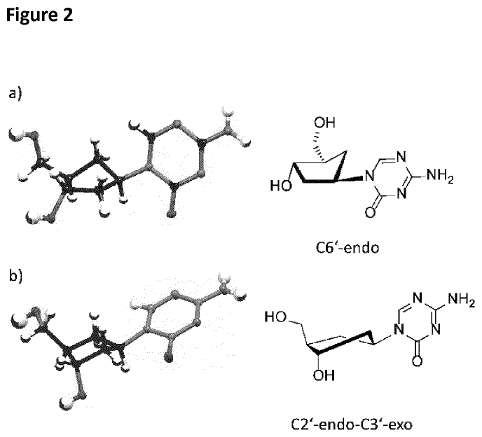

The cAzadC monocrystals were analysed by X-ray crystallography. Table 1 shows the solved structure of cAzadC 1 in two different cyclopentane conformations in the crystal. One conformer adopts a C6′-endo (P=88.2°, max=47.8°) conformation (FIG. 2a), while the second exists as the C2′-endo-C3′-exo (South, P=150.8°, max=45.4°) conformer (FIG. 2b). The latter conformation is typical for 2′-deoxynucleosides in DNA.

Further figures were generated with the program Mercury 3.5.1. (Cambridge Crystallographic Data Center). The data are deposited under CCDC 1910952.

TABLE 1
Crystallographic data for cAzadC

| net formula | C₉H₁₄N₄O₃ | transmission factor | 0.9087- |
|---|---|---|---|
| | | range | 0.9580 |
| Mᵣ/g mol⁻¹ | 226.24 | refls. measured | 34579 |
| crystal size/mm | 0.100 × 0.080 × | Rᵢₙₜ | 0.0373 |
| | 0.030 | | |
| T/K | 100(2) | mean σ(/)// | 0.0197 |
| radiation | MoKα | θ range | 3.031-25.40 |
| diffractometer | ′Bruker | observed refls. | 3474 |
| | D8Venture′ | | |
| crystal system | orthorhombic | x, y (weighting | 0.0372, |
| | | scheme) | 0.4868 |
| space group | ′P 21 21 21′ | hydrogen refinement | mixed |
| a/Å | 8.4451(3) | Flack parameter | 0.3(3) |
| b/Å | 11.0958(4) | refls in refinement | 3787 |
| c/Å | 21.9635(8) | parameters | 321 |
| α/° | 90 | restraints | 0 |
| β/° | 90 | R(F_{obs}) | 0.0284 |
| γ/° | 90 | R_{w}(F²) | 0.0699 |
| V/Å³ | 2058.09(13) | S | 1.054 |
| Z | 8 | shift/errorₘₐₓ | 0.001 |
| calc. density/ | 1.460 | max electron density/e | 0.180 |
| g cm⁻³ | | Å⁻³ | |
| μ/mm⁻¹ | 0.112 | min electron density/e | −0.173 |
| | | Å⁻³ | |

absorption correction multi-scan

### Example 3—Test for Hydrolytic Stability

We next investigated the hydrolytic stability of cAzadC 1 in direct comparison to the pharmaceutical AzadC.

Both nucleosides were dissolved in aqueous KH₂PO₄ buffer (100 mM, pH=7.4, 5.5 and 8.5) in 100 mM concentration. The solutions were immediately analysed via HPLC (t=0 h) with subsequent HPLC analyses every 1.5 h or at indicated time points. As stationary phase an EC 250/4 NUCLEOSIL 120-3 C18 (Macherey-Nagel) chromatography column was used. The mobile phase consisted of water (buffer A) and acetonitrile (buffer B) at a flow-rate of 0.5 mL/min as follows: 0→25 min, 0→5% buffer B; 25 min→28 min, 5%→80%; 28 min→38 min, 80%; 38 min→43 min 80%→0%; 43 min→45 min, 0%.

Since one treatment cycle for AzadC goes over four weeks we decided to measure the stability at a time point related to a half cycle (14 d). NMR spectra were measured after keeping the solutions at r.t.

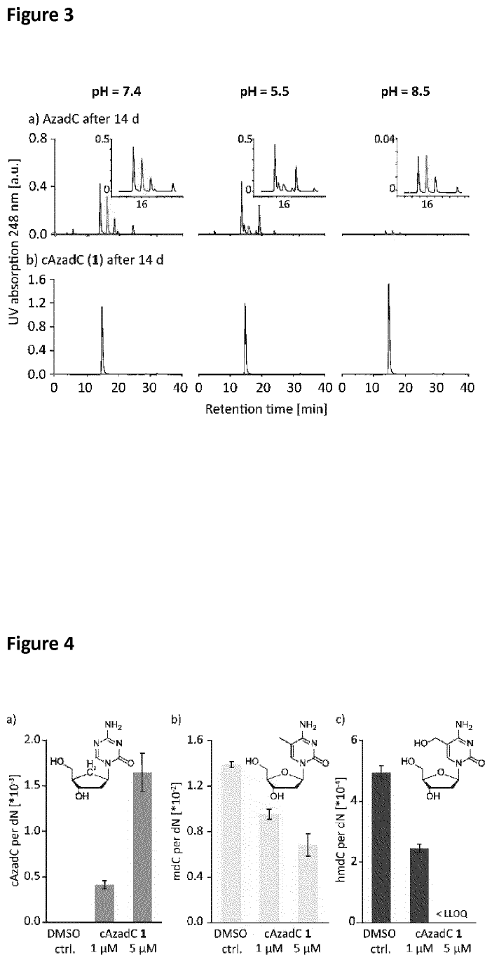

As tumour cells often provide an acidic micro-environment [16], the stability under slightly acidic pH is particularly informative. As evident from the data shown in FIG. 3, the pharmaceutical AzadC strongly degraded within these 14 d. Importantly, at pH=5.5 and at pH=8.5, intact AzadC was only hardly detectable anymore. At physiological pH (7.4), AzadC was still present after 14 d but the level of degradation is dramatic.

In contrast to these results, we observed for cAzadC 1 surprisingly no degradation at all tested pH values, including pH=5.5. This result led to the surprising discovery that the simply O→CH₂ exchange causes a strong remote disarming effect that seems to change the properties of the triazine ring so that reaction with water is stopped.

### Example 4—Test for Biological Functions

### Cell Culture of mESC for cAzadC Treatment

Feeder independent wt J1 (strain 129S4/SvJae) [24] cells were cultured in the presence of serum and LIF as previously described [25]. They were routinely maintained on gelatinized plates in 2i/L medium. For priming experiments, 2i cultures were passaged when applicable in DMEM supplemented with FBS and LIF as above but lacking the inhibitors. For drug treatment, cells were moved into the primed state by removing 2i from the medium. Cells were incubated 2 d in DMEM supplemented with FBS and LIF in 6-well plates (VWR). After splitting, 2×10⁵ cells were transferred into a 6-well plate culture dish and supplemented with either 1 μM (in 0.01% DMSO) or 5 μM cAzadC (in 0.05% DMSO) and treated for 72 h. After removal of the medium and washing the cells with DPBS, they were directly lysed with RLT⁺ buffer as described in a previous publication [18].

### gDNA Isolation, Total Enzymatic Digest and UHPLC-MS²

The gDNA was isolated as described previously [18]. The gDNA was directly subjected to a total enzymatic digest and analyzed using UHPLC-MS² as described in a previous publication [19]. The external calibration curve was generated by serially diluting pure cAzadC [20 pmol, 10 pmol, 5 pmol, 2.5 pmol, 1.25 pmol, 0.625 pmol, 0.3125 pmol] and measuring it in technical triplicates prior to each measurement. Linear regression was done by OriginPro 2016G.

### Results

We investigated if the disarming effect described in Example 3 would influence the biological functions. We used for this purpose mouse embryonic stem cells (mESC) that were primed in serum/Lif as a model system, since mdC levels increase from naïve to primed state [17]. We added cAzadC 1 in two different concentrations (1 μM and 5 μM) to mESC that have been primed for 48 h and allowed the cells to further proliferate under priming conditions in the presence of cAzadC 1 for additional 72 h. After the 72 h, we harvested the cells, isolated the DNA and digested the DNA down to the nucleoside level using our described protocol [18].

The levels of mdC were finally precisely quantified using isotope dilution UHPLC-MS². To this end, isotopically labelled standards of the nucleosides were spiked-in for exact quantification [19, 18]. In addition to mdC, we quantified the levels of 5-hydroxymethyl-2′-deoxycytidine (hmdC), which is formed from mdC by the action of TET enzymes [20-21]. The absolute levels of hmdC are in mESC more than ten times lower than the mdC levels [20, 22]. The consequence is that even after a substantial reduction of mdC, there should be sufficient mdC to keep the hmdC levels constant. The question if and by how much the hmdC level is affected can therefore inform us about how epigenetic reprogramming is organized.

Parallel to the quantification of mdC and hmdC we also quantified to which extend cAzadC 1 itself was incorporated into the genome of the mESC. Detection of AzadC in the genome of treated cells is only possible after treatment of the DNA with NaBH₄. Application of NaBH₄ reduces the C(5)=C(6) double bond, which stabilizes the compound so that its quantification becomes possible [23, 19]. To our delight, we noted that the stability of cAzadC 1 allowed its quantification without this pre-treatment. We also noted that the applied enzymatic digestion protocol allowed to digest genomic DNA (gDNA) even in the presence of large amounts of cAzadC 1. Taken together, quantification of cAzadC 1 by UHPLC-MS² using an external calibration curve was possible in parallel to quantification of canonical and epigenetic bases.

At 1 μM cAzadC 1 concentration, we detected a cAzadC 1 level of 5×10⁻⁴ cAzadC per dN (FIG. 4a). This amounts to almost 3 million cAzadC nucleotides integrated into the genome. At the higher concentration of 5 μM cAzadC 1, the level increased 3-fold to 1.7×10⁻³ cAzadC per dN and consequently to more than 8 million integrated cAzadCs per genome. Compared to the incorporation of AzadC, which reaches 1.2×10⁻³ AzadC per dN, when applied with 1 μM [19], the levels of cAzadC 1 reaches about a third of this level. The data clearly show that the carbocyclic version of AzadC (cAzadC 1) is incorporated and that it reaches in the genome finally comparable levels at 5 μM concentration.

Importantly, after exposing the mESC for 72 h at 1 M cAzadC in the medium, we detected a reduction of the mdC values by almost 30% (FIG. 4b). At 5 M concentration in the medium, the mdC levels dropped even to about 50% of the original value. A decrease to 50% is observed for AzadC as well. Here, however, the 50%-reduction is reached faster (24 h) and already with lower AzadC concentration (1 μM) [19].

The data show that the carbocyclic version cAzadC 1 needs simply more time to affect the mdC levels by the same amount. We believe that this effect is caused by a potentially slower conversion of cAzadC 1 into the triphosphate. The slower kinetics of cAzadC 1, however, is not necessarily a disadvantage given the long treatment times that are applied in the clinic.

Very interesting is also the discovery that the hmdC levels were reduced to about 50% already in the 1 μM experiment. At 5 μM, we were even unable to detect hmdC above background levels using 0.5 μg of genomic DNA. The result shows that the hmdC level dropped even faster than the mdC levels, although hmdC is more than ten times less abundant in the genome. This result is interesting. It indicates that hmdC might be potentially predominantly generated in the mdC maintenance process during cell division. We see here that compound cAzadC 1 is a perfect tool molecule that now allows to gain further insight into the interplay between methylation of dC to mdC and oxidation of mdC to hmdC. With the new compound cAzadC 1 in hand we can cleanly correlate demethylation of the genome with the corresponding cellular effects without compromising DNA damaging effects. Finally, cAzadC 1 may not only be a valuable tool compound but even a next generation epigenetic pharmaceutical.

### SUMMARY

We show that replacement of the in-ring O-atom by a CH₂-unit stabilizes the pharmaceutical so that its nucleophilic reaction with water is stopped. The new nucleoside cAzadC 1 is accepted by the phosphorylating enzymes in cells and the corresponding cAzadC-triphosphates are efficiently incorporated into the genome. cAzadC 1 is incorporated in the genome with several million nucleotides and it causes the mdC level to decrease to 70% relative to the control levels.

### Example 5—Synthetic Procedures for cAzaC (20)

### Tert-Butyl-(1R,4S,5R,6S)-5,6-dihydroxy-3-oxo-2-azabicyclo[2.2.1]heptane-2-carboxylate 12

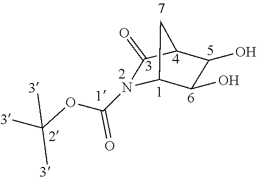

Boc-protected Vince lactam 11 [30] (11.66 g, 55.7 mmol, 1.0 eq.) was dissolved in 214 mL THF, 92 mL tert-butanol and 31 mL H₂O 22.59 g N-methylmorpholino-N -oxide (167.2 mmol, 3.0 eq.) and 1.03 g K₂OsO₄ dihydrate (2.79 mmol, 5 mol %) were added and the mixture was stirred at room temperature for 16 h. Subsequently, 200 mL water and 35.1 g Na₂SO₃ (278.5 mmol, 5.0 eq.) were added and the mixture was stirred for another 1 h at room temperature. Then, the reaction mixture was extracted with EtOAc (3×300 mL). Combined organic layers were washed with saturated aq. NaCl (300 mL) and dried over Na₂SO₄. After evaporation of the solvents in vacuo the residue was purified by silica gel column chromatography (CH₂Cl₂:MeOH 40:1→CH₂Cl₂:MeOH 30:1) to yield 2.91 g of the exo-bishydroxylated product 12 (12.0 mmol, 22%) as a yellow foam.

¹H-NMR (400 MHz, CDCl₃): δ/ppm=4.35-4.32 (m, 1H, 1-H), 4.23 (d, J=6.0 Hz, 1H, 5-H), 4.08 (d, J=6.1 Hz, 1H, 6-H),3.89-3.80 (br s, —OH), 3.78-3.68 (br s, —OH) 2.82-2.76 (m, 1H, 4-H), 2.09 (dt, J=10.9, 1.5 Hz, 1H, 7-H), 1.97 (m, 1H, 7-H), 1.51 (s, 9H, 3′-H).

¹³C-NMR (101 MHz, CDCl₃): δ/ppm=173.30 (3-C), 149.20 (1′-C), 83.82 (2′-C), 70.57 (6-C), 68.14(5-C), 62.29 (1-C), 53.74 (4-C), 31.99 (7-C), 28.16 (3′-C).

IR (ATR): v (cm⁻¹)=3415 (w), 2979 (w), 1777 (m), 1714 (m), 1457 (w), 1394 (w), 1368 (m), 1350 (m), 1326 (m), 1297 (m), 1256 (m), 1144 (s), 1081 (s), 1043 (w), 1031 (w), 1016 (m), 947 (s), 902 (w), 838 (w), 776 (m), 733 (s), 702 (m).

R_{f} (CH₂Cl₂:MeOH 20:1): 0.24.

### Tert-Butyl-((1R,2S,3R,4R)-2,3-dihydroxy-4-(hydroxymethyl)-cyclopentyl) -carbamate 13

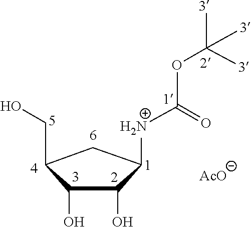

A solution of bishydroxylated lactam 12 (2.90 g, 11.9 mmol, 1.0 eq.) in 40 mL dry MeOH was cooled to 0° C. and NaBH₄ (2.25 g, 59.61 mmol, 5.0 eq.) was added in 5 portions over 15 min. The mixture was further stirred at 0° C. for 45 min, warmed to room temperature and stirred at room temperature for another 1 h. Subsequently, 100 mL acetic acid (10% v/v in MeOH) and silica gel were added and the mixture was concentrated to dryness in vacuo. Purification via silica gel column chromatography (dry load, CH₂Cl₂:MeOH 5:1→CH₂Cl₂:MeOH 4:1) and coevaporation with dry toluene (2×150 mL) yielded product 313 as its acetate salt (2.96 g, 9.6 mmol, 81%) as a slightly brown oil.

¹H-NMR (400 MHz, DMSO-d6): δ/ppm=6.70 (d, J=7.9 Hz, 1H, NH), 3.65-3.49 (m, 2H, 1-H, 3-H), 3.52 (dd, J=6.3, 5.2 Hz, 1H, 2-H), 3.37 (dd, J=10.5, 5.8 Hz, 1H, 5-H), 3.30 (dd, J=10.5, 6.2 Hz, 1H, 5-H), 1.98 (dt, J=13.1, 8.4 Hz, 1H, 6-H), 1.86-1.77 (m, 4H, 4-H, OAc), 1.37 (s, 9H, 3′-H), 1.04-0.90 (m, 1H, 6-H).

¹³C-NMR (101 MHz, DMSO-d6): δ/ppm=173.28 (OAc-C1), 155.23 (C-1′), 77.37 (2′-C), 75.93 (2-C), 72.07 (1-C), 63.02 (5-C), 55.07 (3-C), 44.94 (4-C), 30.61 (6-C), 28.30 (3′-C), 22.49 (OAc-C2).

R_{f} (CH₂Cl₂:MeOH 8:1): 0.43.

### Tert-Butyl-((1R,2S,3R,4R)-2,3-bis(benzyloxy)-4-((benzyloxy)methyl) -cyclopentyl)-carbamate 14

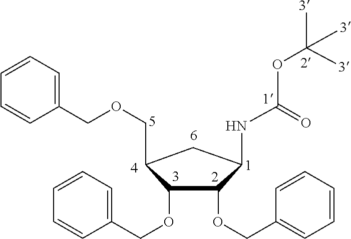

A solution of cyclopentane 13 (2.93 g, 9.5 mmol, 1.0 eq.) in 67 mL dry DMF was cooled to 0° C. and NaH (1.57 g, 39.3 mmol, 4.1 eq., 60% in mineral oil) was added in 3 portions. After stirring the mixture for 15 min at 0° C. 5.31 mL BnBr (44.6 mmol, 4.7 eq,) were added slowly and the reaction mixture was stirred for 2.5 h at 0° C. and for 3 h at room temperature. Then, 220 mg TBAI (0.6 mmol, 6 mol %) and another 3.18 mL BnBr (26.8 mmol, 2.8 eq.) were added and the mixture was warmed to 80° C. for 3.5 h. Subsequently, the mixture was cooled to room temperature, poured in 300 mL saturated aq. NaHCO₃ and extracted with CH₂Cl₂ (3×300 mL). Combined organic layers were washed with saturated aq. NaHCO₃ (300 mL), saturated aq. NaCl (300 mL), dried over anhydrous Na₂SO₄ and concentrated in vacuo at 50° C. The residue was purified by column chromatography (iHex:EtOAc 10:1→iHex:EtOAc 8:1→iHex:EtOAc 7:1→iHex:EtOAc 6:1→iHex:EtOAc 4:1) to yield the tribenzyl-protected product 14 (2.47 g, 4.8 mmol, 51%) a colorless solid wax.

¹H-NMR (400 MHz, CDCl₃): δ/ppm=7.45-7.09 (m, 15H, 3×OCH₂Ph), 4.95 (s, 1H, NH), 4.76-4.25 (m, 6H, 3×OCH₂Ph), 4.15-3.90 (m, 1H, 1-H), 3.82 (dd, J=6.7, 4.4 Hz, 1H. 3-H), 3.79-3.63 (m, 1H, 2-H), 3.57-3.34 (m, 2H, 5-H), 2.51-2.24 (m, 2H, 4-H, 6-H), 1.50-1.34 (m, 9H, 3′-H), 1.31-1.19 (m, 1H, 6-H).

¹³C-NMR (101 MHz, CDCl₃): δ/ppm=155.09 (1′-C), 138.48 (Ar—C), 138.43 (Ar—C), 138.13 (Ar—C), 128.44 (Ar—C), 128.30 (Ar—C), 128.24 (Ar—C), 128.03 (Ar—C), 127.88 (Ar—C), 127.82 (Ar—C), 127.70 (Ar—C), 127.54 (Ar—C), 127.51 (Ar—C), 81.27 (2-C), 79.29 (3-C), 79.13 (2′-C), 73.30 (OCH₂-Ph), 71.52 (OCH₂-Ph), 71.02 (OCH₂-Ph), 70.41 (5-C), 52.85 (1-C), 41.49 (1-C), 30.34 (6-C), 28.46 (3′-C).

HRMS (ESI⁺): calculated for C₃₂H₄₀NO₅⁺ [M+H]⁺ 518.2901; found: 518.2911.

IR (ATR): v (cm⁻¹)=3331 (br w), 3029 (w), 2974 (w), 2867 (w), 1705 (s), 1495 (m), 1453 (m), 1390 (w), 1364 (s), 1245 (m), 1165 (s), 1092 (s), 1071 (s), 1027 (m), 732 (s), 695 (s).

R_{f} (iHex:EtOAc 4:1): 0.69.

### (1R,2S,3R,4R)-2,3-Bis(benzyloxy)-4-((benzyloxy)methyl)-cyclopentane-1-amine 15

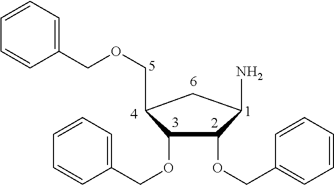

Under Ar atmosphere 1.02 g of Boc-protected cyclopentane 14 (1.97 mmol, 1.0 eq.) were dissolved in 7.0 mL dry CH₂Cl₂, 3.0 mL TFA were added and the mixture was stirred at room temperature for 1 h. The red solution was concentrated to dryness, 20.0 mL saturated aq NaHCO₃ were added and the mixture was stirred at room temperature for 10 min. Then, 60 mL EtOAc were added, phases were separated and the aqueous layer was further extracted with EtOAc (2×60 mL). Combined organic layers were dried over Na₂SO₄ and concentrated to dryness in vacuo to yield deprotected amine 15 as a yellow oil (882 mg, 1.97 mmol, quant.) which was used for the next step without further purification due to its instability towards oxidation.

¹H-NMR (400 MHz, CDCl₃): δ/ppm=7.39-7.26 (m, 15H, 3×OCH₂Ph), 4.63-4.33 (m, 6H, 3×OCH₂Ph), 3.81 (dd, J=5.0, 3.1 Hz, 1H, 3-H), 3.49 (dt, J=9.1, 7.8 Hz, 1H, 1-H), 3.41 (dd, J=9.2, 5.1 Hz, 1H, 5-H), 3.34-3.26 (m, 2H,2-H, 5-H), 2.47-2.34 (m, 1H, 4-H), 2.24-2.13 (m, 1H, 6-H), 1.06-0.94 (m, 1H, 6-H).

¹³C-NMR (101 MHz, CDCl₃): δ/ppm=138.66 (Ar—C), 138.52 (Ar—C), 138.48 (Ar—C), 128.72 (Ar—C), 128.52 (Ar—C), 128.47 (Ar—C), 128.38 (Ar—C), 128.31 (Ar—C), 128.00 (Ar—C), 127.74 (Ar—C), 127.67 (Ar—C), 127.13 (Ar—C), 86.10 (2-C), 78.11 (3-C), 73.21 (OCH₂-Ph), 72.27 (5-C), 71.86 (OCH₂-Ph), 70.89 (OCH₂-Ph), 54.32 (1-C), 41.49 (4-C), 32.19 (6-C).

HRMS (ESI⁺): calculated for C₂₇H₃₂NO₃⁺ [M+H]⁺ 418.2377; found: 418.2378.

### 1-[(1′R,2′S,3′R,4′R)-2′,3′-Bis(benzyloxy)-4′-((benzyloxy)methyl)-cyclopentyl]-methylisobiuret 16

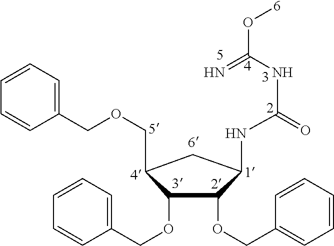

Under Ar atmosphere 882 mg cyclopentyl amine 15 (1.87 mmol, 1.97 mmol, 1.0 eq.) were dissolved in 18.20 mL dry MeCN and 431 mg 2-methyl-1-(1-imidazoylcarbonyl)-isourea ^{[1]} (2.56 mmol, 1.3 eq.) were added. The mixture was refluxed for 2 h at 92° C., subsequently cooled to room temperature and evaporated to dryness. The residue was purified by column chromatography on silica gel (iHex:EtOAc 3:1→iHex:EtOAc 5:2→iHex:EtOAc 2:1→iHex:EtOAc 1:1) to yield methylisobiuret 16 as a colorless oil (886 mg, 1.17 mmol, 87%).

¹H-NMR (400 MHz, CDCl₃): δ/ppm=7.43-7.22 (m, 15H, 3×OCH₂Ph), 5.60 (s, 1H, 1-H), 4.80-4.61 (m, 2H, OCH₂Ph), 4.55-4.41 (m, 3H, OCH₂Ph), 4.32 (d, J=11.8 Hz, 1H, OCH₂Ph), 4.24 (td, J=7.6, 3.4 Hz, 1H, 1′-H), 3.87 (dd, J=6.9, 4.5 Hz, 1H, 3′-H), 3.82 (dd, J=4.5, 3.1 Hz, 1H, 2′-H), 3.61 (s, 2H, 3-H, 5-H), 3.57-3.45 (m, 2H, 5′-H), 2.54-2.39 (m, 2H, 4′-H, 6′-H), 2.17 (s, 3H, 6-H), 1.39-1.31 (m, 1H, 6′-H).

¹³C-NMR (101 MHz, CDCl₃): δ/ppm=207.11 (4-C), 162.27 (2-C), 138.74 (Ar—C), 138.71 (Ar—C), 138.45 (Ar—C), 128.51 (Ar—C), 128.34 (Ar—C), 128.28 (Ar—C), 127.86 (Ar—C), 127.70 (Ar—C), 127.59 (Ar—C), 127.56 (Ar—C), 81.65 (2′-C), 79.79 (3′-C), 73.35 (OCH₂-Ph), 71.64 (OCH₂-Ph), 71.20 (OCH₂-Ph), 70.60 (5′-C), 52.56 (1′-C), 41.68 (4′-C), 31.07 (6-C), 30.57 (6′-C).

HRMS (ESI⁺): calculated for C₃₀H₃₆N₃O₅⁺ [M+H]⁺ 518.2650; found: 518.2652.

IR (ATR): v (cm⁻¹)=3384 (br w), 3028 (w), 2857 (w), 1694 (w), 1635 (s), 1495 (s), 1453 (m), 1364 (m), 1300 (w), 1197 (w), 1096 (s), 1027 (m) 799 (w), 736 (m), 697 (s).

R_{f} (iHex:EtOAc 2:1): 0.37.

### 4-Methoxy-1-[(1′R,2′S,3′R,4′R)-2′,3′-bis(benzyloxy)-4′-((benzyloxy)methyl) -cyclopentyl]-1H-[1,3,5]-triazin-2-one (17) and 4-ethoxy-1-[(1′R,2′S,3′R,4′R) -2′,3′-bis(benzyloxy)-4′-((benzyloxy)rnethyl)-cyclopentyl]-1 H-[1,3,5]-triazin-2-one (18)

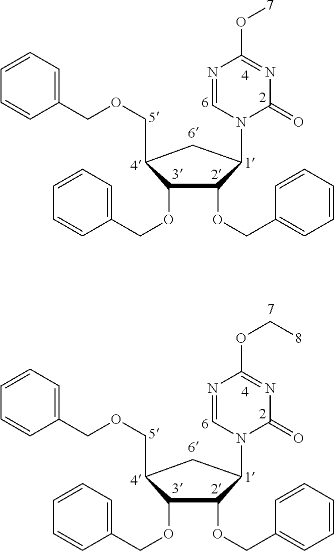

Methylisobiuret 16 (837 mg, 1.62 mmol, 1.0 eq.) was dissolved in 15.30 mL triethylorthoformate and 20 μL TFA were added. The mixture was heated to 90° C. for 3 h, subsequently cooled to room temperature and concentrated in vacuo at 50° C. The residue was coevaporated with dry MeOH and purified by column chromatography on silica gel (iHex:EtOAc 3:1→iHex:EtOAc 5:2→iHex:EtOAc 2:1→iHex:EtOAc 1:1→iHex:EtOAc 1:2) to yield 373 mg ethoxytriazine 18 (0.69 mmol, 43%) as a colorless wax and 389 mg methoxytriazine 17 (0.73 mmol, 46%) as a colorless solid.

Methoxytriazine 17:

¹H-NMR (400 MHz, CDCl₃): δ/ppm=7.96 (s, 1H, 6-H), 7.38-7.17 (m, 15H, 3×OCH₂Ph), 4.70-4.40 (m, 6H, 1′-H, 2.5×OCH₂Ph), 4.36-4.24 (m, 2H, 2′-H, 0.5×OCH₂Ph), 3.98 (s, 3H, 7-H), 3.93 (dd, J=4.9, 3.1 Hz, 1H, 3′-H), 3.54-3.40 (m, 2H, 5′-H), 2.51 (ddt, J=9.8, 4.4, 2.3 Hz, 1H, 4′-H), 2.35 (dt, J=13.3, 9.4 Hz, 1H, 6′-H), 1.82 (ddd, J=13.4, 9.0, 6.7 Hz, 1H, 6′-H).

¹³C-NMR (101 MHz, CDCl₃): δ/ppm=169.87 (4-C), 159.59 (6-C), 154.67 (2-C), 138.16 (Ar—C), 138.14 (Ar—C), 137.63 (Ar—C), 128.63 (Ar—C), 128.60 (Ar—C), 128.51 (Ar—C), 128.24 (Ar—C), 128.20 (Ar—C), 128.17 (Ar—C), 127.90 (Ar—C), 127.86 (Ar—C), 78.78 (2′-C), 77.27 (3′-C), 73.40 (OCH₂-Ph), 72.17 (OCH₂-Ph), 71.41 (OCH₂-Ph), 71.20 (5′-C), 63.34 (1′-C), 55.95 (7-C), 41.14 (4′-C), 26.75 (6′-C).

HRMS (ESI⁺): calculated for C₃₁H₃₄N₃O₅⁺ [M+H]⁺ 528.2493; found: 528.2498.

IR (ATR): v (cm⁻¹)=3029 (w), 2867 (w), 1698 (s), 1613 (s), 1515 (m), 1464 (m), 1453 (m), 1398 (m), 1337 (m), 1265 (w), 1211 (w), 1096 (m), 1070 (m), 1027 (m), 913 (w), 802 (m), 733 (s), 696 (s).

R_{f} (iHex:EtOAc 2:1): 0.27.

Ethoxytriazine 18:

¹H-NMR (400 MHz, CDCl₃): δ/ppm=7.97 (s, 1H, 6-H), 7.39-7.15 (m, 15H, 3×OCH₂Ph), 4.66-4.21 (m, 10H, 1′-H, 2′-H, 7-H, 3×OCH₂Ph), 3.96-3.87 (m, 1H, 3′-H), 3.54-3.36 (m, 2H, 5′-H), 2.56-2.41 (m, 1H, 4′-H), 2.33 (ddd, J=14.5, 9.5, 4.7 Hz, 1H, 6′-H), 1.88-1.74 (m, 1H, 6′-H), 1.25 (q, J=6.9 Hz, 3H, 8-H).

¹³C-NMR (101 MHz, CDCl₃): δ/ppm=169.31 (4-C), 159.53 (6-C), 154.74 (2-C), 138.18 (Ar—C), 138.16 (Ar—C), 137.64 (Ar—C), 128.79 (Ar—C), 128.64 (Ar—C), 128.61 (Ar—C), 128.51 (Ar—C), 128.25 (Ar—C), 128.20 (Ar—C), 128.19 (Ar—C), 127.90 (Ar—C), 127.87 (Ar—C), 79.11 (2′-C), 77.36 (3′-C), 73.46 (OCH₂-Ph), 72.28 (OCH₂-Ph), 71.55 (OCH₂-Ph), 71.20 (5′-C), 65.06 (7-C), 63.33 (1′-C), 41.15 (4′-C), 26.81 (6′-14.24 (8-C).

HRMS (ESI⁺): calculated for C₃₂H₃₆N₃O₅⁺ [M+H]⁺ 542.2650; found: 542.2656.

IR (ATR): v (cm⁻¹)=3064 (w), 3030 (w), 2924 (w), 2962 (w), 1694 (s), 1615 (s), 1514 (m), 1495 (m), 1452 (s), 1361 (m), 1326 (m), 1270 (m), 1206 (w), 1096 (s), 1070 (s), 1027 (m), 803 (w), 734 8s), 697 (s).

R_{f} (iHex:EtOAc 2:1): 0.30.

### 4-Amino-1-[(1′R,2′S,340 R,4′R)-2′,3′-bis(benzyloxy)-4′-((benzyloxy)methyl) -cyclopentyl]-1H-[1,3,5]-triazin-2-one 19

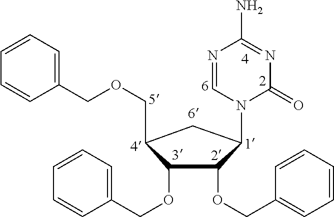

A mixture of ethoxytriazine 18 (358 mg, 0.66 mmol) and methoxytriazine 17 (363 mg, 0.69 mmol) were dissolved in 26.8 mL 7 N NH₃ in MeOH and stirred at room temperature for 3 h. Subsequently, 125 mL H₂O were added to the reaction mixture, which was then extracted with EtOAc (3×200 mL). Combined organic layers were dried over Na₂SO₄ and evaporated to dryness in vacuo yielding 593 mg of the aminated product 19 (1.16 mmol, 86%) as a colorless foam which was used for the next step without further purification.

¹H-NMR (400 MHz, CDCl₃): δ/ppm=7.80 (s, 1H, 6-H), 7.26 (m, 15H, 3×OCH₂Ph), 5.70 (s, 1H, NH), 4.63-4.42 (m, 6H, 1′-H, 2.5×OCH₂Ph), 4.39-4.28 (m, 2H, 2′-H, 0.5×OCH₂Ph), 3.91 (dd, J=5.0, 3.5 Hz, 1H, 3′-H), 3.53-3.42 (m, 2H, 5′-H), 2.59-2.35 (m, 1H, 4′-H), 2.26 (dt, J=13.3, 9.3 Hz, 1H, 6′-H), 1.86 (ddd, J=13.3, 9.2, 7.1 Hz, 1H, 6′-H)

¹³C-NMR (101 MHz, CDCl₃): δ/ppm=165.84 (4-C), 158.30 (6-C), 154.11 (2-C), 138.27 (Ar—C), 138.24 (Ar—C), 137.83 (Ar—C), 128.57 (Ar—C), 128.56 (Ar—C), 128.47 (Ar—C), 128.21 (Ar—C), 128.12 (Ar—C), 128.05 (Ar—C), 127.84 (Ar—C), 127.82 (Ar—C), 127.78 (Ar—C), 78.99 (2′-C), 77.39 (3′-C), 73.30 (OCH₂-Ph), 72.18 (OCH₂-Ph), 71.39 (OCH₂-Ph), 71.3 (5′-C)7, 63.53 (1′-C), 41.21 (4′-C), 26.79 (6′-C).

HRMS (ESI⁺): calculated for C₃₀H₃₃N₄O₄⁺ [M+H]⁺ 513.2496; found: 513.2496.

IR (ATR): v (cm⁻¹)=3331 (br w), 3182 (br w), 3060 (w), 3028 (w), 2925 (w), 2857 (w), 1633 (s), 1506 (m), 1495 8m), 1470 (m), 1452 8s), 1361 (w), 1262 (w), 1092 (s), 1070 (s), 1027 (m), 912 (w), 799 (m), 734 (s), 696 (s).

R_{f} (CH₂Cl₂:MeOH 15:1): 0.42.

### 4-Amino-1-[(1′R,2′S,3′R,4′R)-2′,3′-bishydroxy-4′-(hydroxymethyl) -cyclopentyl]-1H-[1,3,5]-triazin-2-one 20

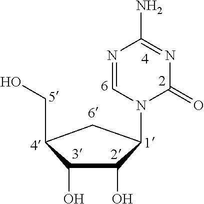

A solution of benzyl protected 5-aza-carbaC 19 (576 mg, 1.12 mmol, 1.0 eq.) was dissolved in 30.8 mL dry CH₂Cl₂ under Ar atmosphere and cooled to −78° C. Then, 5.88 mL BCl₃ (1 M in CH₂Cl₂, 5.88 mmol, 5.25 eq.) were added dropwise, the mixture was stirred 1 h at −78° C. and 2 h at room temperature. Subsequently, 40 mL dry MeOH were added, the mixture was stirred for another 20 min at room temperature and concentrated to dryness in vacuo. The residue was purified by column chromatography on silica gel (dry load, CH₂Cl₂:MeOH 8:1→CH₂Cl₂:MeOH 5:1→CH₂Cl₂:MeOH 4:1→CH₂Cl₂:MeOH 7:3) to yield 228 mg of the deprotected compound 20 (0.94 mmol, 84%) as a colorless solid.

For cell biological application 34.5 mg of the compound were further purified by reversed phase HPLC (0% to 3% MeCN in H₂O in 45 min, flow 5 mL/min) to yield 25.3 mg of HPLC-purified 20 (73%).

¹H-NMR (400 MHz, D₂O): δ/ppm=8.32 (s, 1H, 6-H), 4.57-4.33 (m, 2H, 1′-H, 2′-H), 4.04 (dd, J=5.3, 4.4 Hz, 1H, 3′-H), 3.76-3.57 (m, 2H, 5′-H), 2.29 (dt, J=13.0, 8.2 Hz, 1H, 6′-H), 2.23-2.15 (m, 1H, 4′-H), 1.76-1.59 (m, 1H, 6′-H).

¹³C-NMR (101 MHz, D₂O): δ/ppm=165.45 (4-C), 159.03 (6-C), 156.15 (2-C), 73.12 (2′-C), 71.77 (3′-C), 63.55 (1′-C), 62.90 (5′-C), 44.42 (4′-C), 27.17 (6′-C).

HRMS (ESI⁺): calculated for C₉H₁₅N₄O₄⁺ [M+H]⁺ 243.1088; found: 243.1086.

HRMS (ESI⁻): calculated for C₉H₁₃N₄O₄⁻ [M−H]⁻ 241.0942; found: 241.0941.

R_{f} (CH₂Cl₂:MeOH 7:3): 0.29.

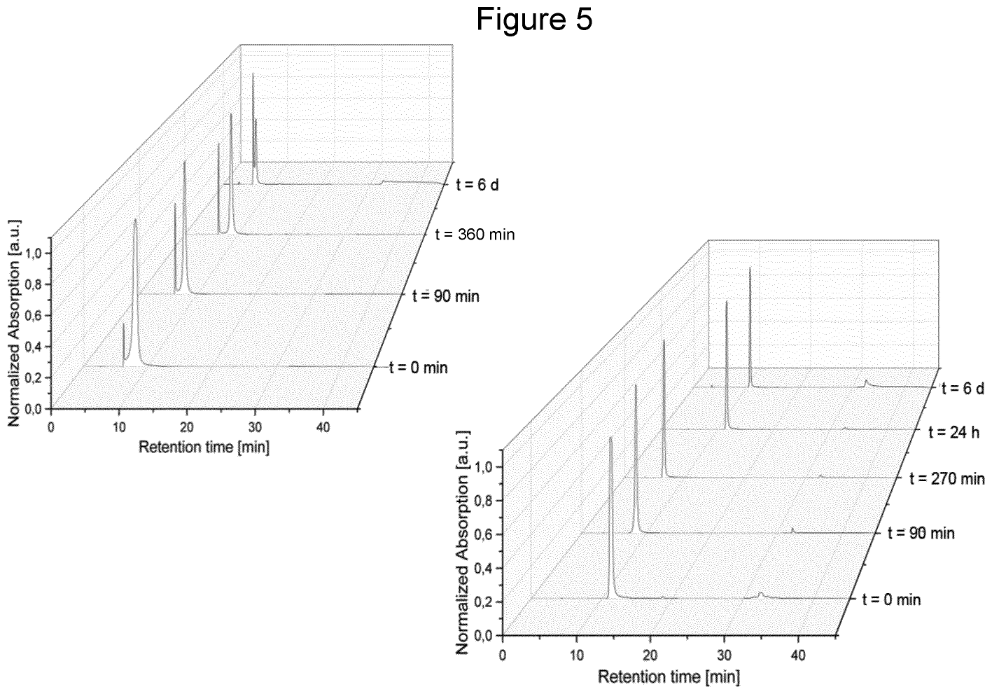

In an HPLC stability test it was shown that the carbacyclic compound cAzaC 20 is much more stable than azacitidine (decitabine) (FIG. 5).

### Example 6

### Synthetic Procedures for a cAzaC Prodrug 24

For an improved delivery into cells we also prepared a 5′-phosphoroamidate prodrug 24 of cAzaC 20.

### 4-Amino-1-((3aS,4R,6R,6aR)-6-(hydroxymethyl)-2,2-dimethyltetrahydro-4H -cyclopenta-[d][1,3]dioxo1-4-yl)-1,3,5-triazin-2(1H)-one 21

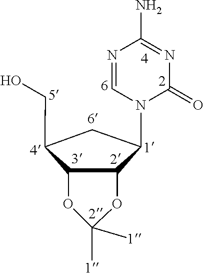

In a heat-dried flask 5-aza-carbaC 20 (64 mg, 264 μmol, 1.0 eq.) was suspended in 6.60 mL dry acetone. p-Toluenesulfonic acid (5 mg, catalytic amount) and 2,2-dimethoxypropane (49 μL, 396 μmol, 1.5 eq.) were added and the mixture was stirred 18 h at room temperature. To the resulting solution 150 μL NEt₃ (1.08 mmol) were added and the mixture was stirred another 10 min at room temperature. Subsequently, the mixture was concentrated to dryness and the obtained residue was purified by column chromatography on silica gel (CH₂Cl₂:MeOH 10:1, containing 1% NEt₃) to yield 81 mg of the isopropylidene -acetal protected compound 21 (182 μmol, 69%, adduct with 1.6 eq. NEt₃) as a colorless solid.

¹H-NMR (400 MHz, D₃COD): δ/ppm=8.29 (s, 1H, 6-H), 4.94 (dd, J=7.0, 4.9 Hz, 1H, 2′-H), 4.62-4.39 (m, 2H, 1′-H, 3′-H), 3.73-3.51 (m, 2H, 5′-H), 3.21 (q, J=7.3 Hz, 10H, N(CH₂CH₃)₃), 2.32-2.07 (m, 3H, 4′-H, 6′-H), 1.50 (s, 3H, 1″-H), 1.32 (t, J=7.3 Hz, 15H, N(CH₂CH₃)₃), 1.29 (s, 3H, 1″-H).

¹³C-NMR (101 MHz, D₃COD): δ/ppm=167.87 (4-C), 159.88 (6-C), 156.66 (2-C), 114.31 (2″-C), 83.95 (2′-C), 82.98 (3′-C), 66.26 (1′-C), 64.17 (5′-C), 47.88 (N(CH₂CH₃)₃), 47.81 (4′-C), 33.72 (6′-C), 27.97 (1″-C), 25.53 (1″-C), 9.23 (N(CH₂CH₃)₃).

HRMS (ESI⁺): calculated for C₁₂H₁₉N₄O₄⁺ 283.1401 [M+H]⁺; found: 283.1397.

IR (ATR): v (cm⁻¹)=3346 (br w(, 2983 (w), 2658 (w), 1634 (s), 1472 (s), 1210 (m), 1160 (m), 1065 (m), 868 (w), 800 (m), 682 (w).

R_{f} (CH₂Cl₂:MeOH 6:1): 0.36.

### Isopropyl((((3aR,4R,6R,6aS)-6-(4-amino-2-oxo-1,3,5-triazin-1(2H)-yl)-2,2-dimethyltetrahydro-4H-cyclopenta[d][1,3]dioxol-4-yl)methoxy)(phenoxy)phosphoryl)-L-alaninate 22

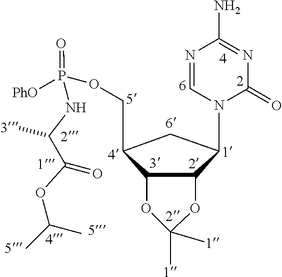

Protected 5-aza-carbaC 21 (69 mg, 155 μmol, 1.0 eq., as a 1.6 eq. NEt₃-adduct) was suspended in 2.40 mL dry THF and cooled to −78° C. Then 855 μL tBuMgCl-solution (1.0 M in THF, 855 μmol, 5.5 eq.) were added, the mixture was directly warmed to 0° C. and stirred for 30 min at this temperature. Subsequently, the mixture was again cooled to −78° C. and 538 μL of a solution of phosphoroxyaryl to chloridate [31] 23 (1.0 M in THF, 538 μmol, 3.5 eq.) were added dropwise. The reaction mixture was allowed to warm to room temperature slowly and stirred for 17 h. The resulting clear yellow solution was quenched by addition of H₂O (20 mL) and the aqueous layer was extracted with EtOAc (3×25 mL). Combined organic phases were washed with saturated aq. NaCl (25 mL), dried over anhydrous Na₂SO₄ and concentrated to dryness in vacuo. The residue was purified by column chromatography on silica gel (CH₂Cl₂:MeOH 60:1→CH₂Cl₂:MeOH 50:1→CH₂Cl₂:MeOH 40:1) to yield 83 mg of product 12 (78 μmol, 51%, adduct with 5.0 eq. NEt₃) as a mixture of both diastereoisomers on P as a colourless solid.

¹H-NMR (800 MHz, CDCl₃): δ/ppm=8.05 (2×s, 1H), 7.32-7.27 (m, 2H, OPh), 7.23-7.17 (m, 2H, OPh), 7.15-7.07 (m, 1H, OPh), 5.04-4.87 (m, 2H, 2′-H, 4′″-H), 4.59-4.54 (m, 1H, 3′-H), 4.31-4.13 (m, 3H, 1′-H, 5′-H), 4.02-3.93 (m, 1H, 2′″-H), 3.78 (m, 1H, NH), 3.09 (q, J=7.4 Hz, 30H, N(CH₂CH₃)₃), 2.53-2.17 (m, 3H, 4′-H, 6′-H), 1.49-1.47 (m, 3H, 1″-H), 1.39 (t, J=7.3 Hz, 45H, N(CH₂CH₃)₃), 1.37-1.34 (m, 3H, 3″-H), 1.26-1.24 (m, 3H, 1″-H), 1.21 -0.18 (m, 6H, 5″′-H).

¹³_{C}-NMR (201 MHz, CDCl₃): δ/ppm=173.10 (1″′-C), 165.39 (4-C), 158.57 (6-C), 153.13 (O—C_{Ar}), 150.83 (2-C), 129.73 (CH_{Ar}), 124.99 (CH_{Ar}), 120.45 (CH_{Ar}), 113.57 (2″-C), 82.42 (2′-C), 81.17 (3′-C), 69.28 (4″′-C), 67.50 (1′-C), 65.93 (5′-C), 50.50 (2″′-C), 45.91 (N(CH₂CH₃)₃), 44.93 (4′-C), 32.34 (6′-C), 29.79 (5″′-C), 27.73 (1″-C), 25.36 (1″-C), 21.72 (5″′-C), 21.13 (3″′-C), 8.75 (N(CH₂CH₃)₃).

³¹P-NMR (162 MHz, CDCl₃): δ/ppm=2.46, 2.36.

HRMS (ESI⁺): calculated for C₂₄H₃₅N₅O₈P⁺ 552.2218 [M+H]⁺; found: 552.2211.

HRMS (ESI⁻): calculated for C₂₄H₃₃N₅O₈P⁺ 550.2072 [M−H]⁻; found: 550.2074.

IR (ATR): v (cm⁻¹)=3379 (m), 2980 (s), 2946 (m), 2606 (s), 2498 (s), 1640 (s), 1476 (s), 1398 (m), 1383 (m), 1212 (s), 1158 (m), 1107 (w), 1070 (m), 1037 (s), 929 (m), 802 (w), 730 (m).

R_{f} (CH₂Cl₂:MeOH 20:1): 0.25.

### Isopropyl((((1R,2R,3S,4R)-4-(4-amino-2-oxo-1,3,5-triazin-1(2H)-yl)-2,3-dihydroxy-cyclopentyl)methoxy)(phenoxy)phosphoryl)-L-alaninate 24

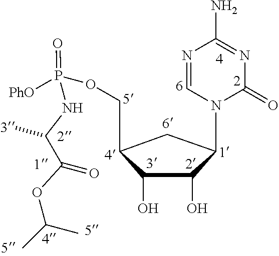

To isopropylidene acetal protected compound 22 (38 mg, 69 μmol, 1.0 eq.) 1.0 mL 80% TFA in ddH₂O was added and the mixture was stirred 20 min at 30° C. Then, the mixture was poured into 5.0 mL ddH₂O and lyophilized. The resulting residue was dissolved in a mixture of 500 μL MeCN, 500 μL DMSO and 4.0 mL ddH₂O containing 0.1% TFA, the turbid solution was filtered and purified by reversed-phase HPLC (5% MeCN in ddH₂O cont. 0.1% TFA to 60% MeCN in ddH₂O cont. 0.1% TFA in 45 min, flow 5 mL/min) to yield 10.4 mg of the target compound 24 (20 μmol, 29%) as a colorless solid.

¹H-NMR (400 MHz, D₃CCN): δ/ppm=8.11 (s, 1H), 7.50 (s, 1H, NH₂), 7.41-7.29 (m, 2H, OPh), 7.25-7.15 (m, 3H, OPh), 6.61 (s, 1H, NH₂), 4.98-4.88 (m, 1H, 4″-H), 4.39-4.23 (m, 3H, 1′-H, 2′-H, NH), 4.20-4.07 (m, 2H, 5′-H), 3.97 (dt, J=5.2, 3.8 Hz, 1H, 3′-H), 3.93-3.82 (m, 1H, 2″-H), 2.33-2.09 (m, 2H, 4′-H, 6′-H), 1.83-1.67 (m, 1H, 6′-H), 1.30-1.26 (m, 3H, 3″-H), 1.19 (dt, J=6.2, 3.4 Hz, 6H, 5″-H).

¹³C-NMR (101 MHz, D₃CCN): δ/ppm=174.00 (1″-C), 165.92 (4-C), 159.92 (6-C), 153.84 (2-C), 151.97 (O—C_{Ar}), 130.62 (CH_{Ar}), 125.75 (CH_{Ar}), 121.36 (CH_{Ar}), 121.31 (CH_{Ar}), 74.12 (2′-C), 72.50 (3′-C), 69.64 (4″-C), 68.53 (5′-C), 64.96 (1′-C), 51.44 (2″-C), 44.44 (4′-C), 28.08 (6′-C), 21.89 (5″-C), 21.81 (5″-C), 20.80 (3″-C).

³¹P-NMR (162 MHz, D₃CCN): δ/ppm=3.08, 2.92.

HRMS (ESI⁺): calculated for C₂₁H₃₁N₅O₈P⁺ 512.1905 [M+H]⁺; found: 512.1901.

HRMS (ESI⁻): calculated for C₂₁H₂₉N₅O₈P⁻ 510.1759 [M−H]⁻; found: 510.1757.

### LIST OF REFERENCES

[1] M. Daskalakis, T. T. Nguyen, C. Nguyen, P. Guldberg, G. Kohler, P. Wijermans, P. A. Jones, M. Lubbert, Blood 2002, 100, 2957-2964.

[2] H. Kantarjian, J. P. Issa, C. S. Rosenfeld, J. M. Bennett, M. Albitar, J. DiPersio, V. Klimek, J. Slack, C. de Castro, F. Ravandi, R. Helmer, 3rd, L. Shen, S. D. Nimer, R. Leavitt, A. Raza, H. Saba, Cancer 2006, 106, 1794-1803.

[3] C. Stresemann, F. Lyko, Int. J. Cancer 2008, 123, 8-13.

[4] Y. Koh, Y. A. Kim, K. Kim, J.-A. Sim, S.-S. Yoon, S. M. Park, Y. H. Yun, Blood 2016, 128, 2381-2381.

[5] M. Nieto, P. Demolis, E. Béhanzin, A. Moreau, I. Hudson, B. Flores, H. Stemplewski, T. Salmonson, C. Gisselbrecht, D. Bowen, F. Pignatti, Oncologist 2016, 21, 692-700.

[6] S. S. Smith, B. E. Kaplan, L. C. Sowers, E. M. Newman, Proc. Natl. Acad. Sci. 1992, 89, 4744-4748.

[7] G. G. Wilson, R. J. Roberts, S. Kumar, J. Posfai, M. Sha, S. Klimasauskas, X. Cheng, Nucleic Acids Res. 1994, 22, 1-10.

[8] A. Bird, Genes Dev. 2002, 16, 6-21.

[9] R. J. Klose, A. P. Bird, Trends Biochem. Sci. 2006, 31, 89-97.

[10] R. Jüttermann, E. Li, R. Jaenisch, Proc. Natl. Acad. Sci. 1994, 91, 11797-11801.

[11] S. Gabbara, A. S. Bhagwat, Biochem. J. 1995, 307, 87-92.

[12] M. Ober, H. Müller, C. Pieck, J. Gierlich, T. Carell, J. Am. Chem. Soc. 2005, 127, 18143-18149.

[13] H. Müller, T. Carell, Eur. J. Org. Chem. 2007, 2007, 1438-1445.

[14] F. Büsch, J. C. Pieck, M. Ober, J. Gierlich, G. W. Hsu, L. S. Beese, T. Carell, Chem. Eur. J. 2008, 14, 2125-2132.

[15] T. H. Gehrke, U. Lischke, K. L. Gasteiger, S. Schneider, S. Arnold, H. C. Müller, D. S. Stephenson, H. Zipse, T. Carell, Nat. Chem. Bio. 2013, 9, 455.

[16] Y. Kato, S. Ozawa, C. Miyamoto, Y. Maehata, A. Suzuki, T. Maeda, Y. to Baba, Cancer Cell Int. 2013, 13, 89.

[17] S. Takahashi, S. Kobayashi, I. Hiratani, Cell. Mol. Life Sci. 2018, 75, 1191-1203.

[18] F. R. Traube, S. Schiffers, K. Iwan, S. Kellner, F. Spada, M. Müller, T. Carell, Nat. Protoc. 2019, 14, 283-312.

[19] S. Schiffers, T. M. Wildenhof, K. Iwan, M. Stadlmeier, M. Müller, T. Carell, Helv. Chim. Acta 2019, 102, e1800229.

[20] M. Tahiliani, K. P. Koh, Y. Shen, W. A. Pastor, H. Bandukwala, Y. Brudno, S. Agarwal, L. M. Iyer, D. R. Liu, L. Aravind, A. Rao, Science 2009, 324, 930-935.

[21] S. Ito, L. Shen, Q. Dai, S. C. Wu, L. B. Collins, J. A. Swenberg, C. He, Y. Zhang, Science 2011, 333, 1300-1303.

[22] M. Münzel, D. Globisch, T. Carell, Angew. Chem. Int. Ed. Engl. 2011, 50, 6460-6468.

[23] A. Unnikrishnan, A. N. Q. Vo, R. Pickford, M. J. Raftery, A. C. Nunez, A. Verma, L. B. Hesson, J. E. Pimanda, Leukemia 2018, 32, 900-910.

[24] E. Li, T. H. Bestor, R. Jaenisch, Cell 1992, 69, 915-926.

[25] T. Pfaffeneder, B. Hackner, M. Truss, M. Münzel, M. Müller, C. A. Deiml, C. Hagemeier, T. Carell, Angew. Chem. Int. Ed. Engl. 2011, 50, 7008-7012.

[26] Ravandi, F.; Kantarjian, J. E.; Issa, S.; Jabbour, S.; Santos, G.; McCue, D.; Garcia-Manero, F. P. S.; Pierce, E.; O'Brien, J. P.; Cortes, J. E.; Ravandi, F. (2010). “Feasibility of Therapy with Hypomethylating Agents in Patients with Renal Insufficiency”. Clinical Lymphoma, Myeloma & Leukemia. 10 (3): 205-210.

[27] Dunn, J; Thabet, S; Jo, H (July 2015). “Flow-Dependent Epigenetic DNA Methylation in Endothelial Gene Expression and Atherosclerosis”. Arteriosclerosis, Thrombosis, and Vascular Biology. 35 (7): 1562-9.

[28] Hecker, S. J.; Erion, M. D. (2008), “Prodrugs of phosphates and phosphonates”. J. Med. Chem 51: 2328-2345.

[29] Wiemer, J.; Wiemer D.F. (2015), “Prodrugs of phosphonates and phosphates: Crossing the membrane barrier”. Top. Curr. Chem. 360: 115-160.

[30] Wildenhof, T. M., Schiffers, S., Traube, F. R., Mayer, P., Carell, T., Angew. Chem. Int. Ed. 2019, 58, 12984-12987.

[31] Davey, S. M., Malde, R., Mykura, R. C., Baker, A. T., Taher, T. E., Le Duff, C. S., Willcox, B. E., Mehellou, Y., J. Med. Chem. 2018, 61, 2111-2117.

1. A compound of Formula I 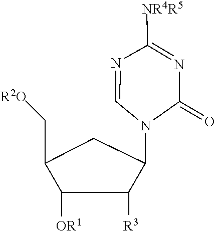
wherein R¹ is H, a free or protected, modified or unmodified phosphate group or a hydroxyl-protecting group,
R² is H, a free or protected, modified or unmodified phosphate group, or a hydroxyl-protecting group,
R³ is H, F, CH3, CH2F, CHF2, CF3, OH or OR⁶ wherein R⁶ is a hydroxyl-protecting group,
R⁴ and R⁵ each are H or form an amino-protecting group,
or a salt thereof.

2. The compound of claim 1 wherein each phosphate group is independently selected from:
(i) a free or protected unmodified phosphate, wherein the phosphate group is a monophosphate, diphosphate or triphosphate group, and
(ii) a free or protected modified phosphate, e.g. monophosphate, diphosphate or triphosphate group, wherein the modified phosphate group is selected from a phosphonate, phosphoramidate or phosphorothioate group.

3. The compound of claim 1 wherein R¹ is H.

4. The compound of claim 1 wherein R² is H.

5. The compound of claim 1 wherein R³ is H or OH.

6. The compound of claim 1 wherein R⁴ and R⁵ are H.

7. The compound of claim 1 wherein R¹, R², R³, R⁴ and R⁵ each are H, or wherein R¹, R², R⁴ and R⁵ each are H and R³ is OH or OR⁶.

8. The compound of claim 1 wherein R¹, R², R³, R⁴ and R⁵ each are H.

9. The compound of claim 1 wherein R¹, R², R⁴ and R⁵ each are H and R³ is OH.

10. The compound of claim 1 or a pharmaceutically acceptable salt thereof for use in medicine.

11. The compound of claim 1 or a pharmaceutically acceptable salt thereof for the treatment of a hyperproliferative disorder.

12. The compound of claim 1 or a pharmaceutically acceptable salt thereof for the treatment of cancer.

13. The compound of claim 1 or a pharmaceutically acceptable salt thereof for the treatment of acute myeloid leukemia (AML), or a myelodysplastic syndrome (MDS).

14. The compound of claim 1 or a pharmaceutically acceptable salt thereof for the treatment of atherosclerosis or renal insufficiency.

15. An in vitro use of the compound of claim 1 for inhibiting the methylation of nucleic acids, particularly of DNA.

16. The use of claim 15 for an epigenetic analysis.

17. A method for the treatment of a hyperproliferative disorder, comprising administering a therapeutically effective dose of the compound of claim 1 or a pharmaceutically acceptable salt thereof to a subject in need thereof, particularly to a human subject.
